(19) United States
(12) Patent Application Publication
(10) Pub. No.: US 2010/0285032 A1
(43) Pub. Date: Nov. 11, 2010

(54) ESTROGEN RECEPTOR-RELATED RECEPTOR GAMMA (ERR GAMMA) IN BONE AND CARTILAGE FORMATION AND METHODS AND COMPOSITIONS RELATING TO SAME

(76) Inventors: Jane E. Aubin, Toronto (CA); Marco Cardelli, Toronto (CA); Ralph A. Zirngibl, Georgetown (CA)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(21) Appl. No.: 12/663,457

(22) PCT Filed: Jun. 9, 2008
(86) PCT No.: PCT/CA08/01097

§ 371 Date: Jul. 13, 2010

Related U.S. Application Data
(60) Provisional application No. 60/942,653, filed on Jun. 7, 2007.

(51) Int. Cl. A61K 39/395; C12N 5/071; A61K 31/7088; A61K 38/16; A61K 31/155; A61P 19/00

(52) U.S. Cl. 424/172.1; 435/325; 514/44 A; 514/16.7; 514/17.1; 514/632

(57) ABSTRACT

ERRγ is expressed in bone and cartilage in vivo and osteoblastic and chondrocytic cells in vitro. ERRγ is a transcriptional activator of an osteoblast-associated osteopontin (OPN) and chondrocyte-associated (Sox9) gene in osteoblasts and chondrocytes respectively. Knockdown of ERRγ expression by antisense oligonucleotide strategies in osteoblastic cell cultures reduces alkaline phosphatase activity. Together these findings indicate that ERRγ is expressed in and plays a functional role in bone and cartilage. The results also indicate that agonists and antagonists of ERRγ may be useful as therapeutic agents in a wide variety of diseases affecting bones and joints.

This application claims priority from U.S. Provisional Patent Application No. 60/942,653, filed Jun. 7, 2007, and incorporated herein by reference.

### FIELD OF THE INVENTION

The present invention relates to methods and pharmaceutical preparations for modulation of bone and cartilage formation.

### BACKGROUND OF THE INVENTION

In the description which follows, references are made to certain literature citations which are listed at the end of the specification and all of which are incorporated herein by reference.

The nuclear receptors (NRs) are transcription factors comprising both ligand-dependant molecules (e.g, steroid hormone, thyroid hormone, retinoic acid and vitamin D receptors) and a large number of so-called orphan receptors for which ligands have not been identified (Blumberg and Evans, 1998; Robinson-Rechavi et al., 2001). Two of the known ligand-dependent NRs are those involved in estrogen response, i.e., estrogen receptor alpha (ERα) and beta (ERβ) (NR3A1 and NR3A2, respectively, according to the Nuclear Receptors Nomenclature Committee (Committee, 1999)). The first orphan NRs identified were proteins related to ERα and were referred to as estrogen receptor-related receptors (ERRs) (Giguere et al., 1988). ERRα and ERRβ (NR3B1 and NR3B2) were identified by low-stringency screening of cDNA libraries with a probe encompassing the DNA binding domain of human ERα. A third ERR, ERRγ, was identified by yeast two-hybrid screening with the NR cofactor, glucocorticoid receptor-interacting protein 1 (GRIP1) as bait (Hong et al., 1999).

ERRs, like most NRs, are modular domain proteins. The DNA-binding domain of ERRs and ERs is highly conserved, however other parts of the proteins share very little homology (Giguere et al., 1988; Hong et al., 1999). Thus, sequence alignment of ERRs, including ERRα and ERRγ which share high sequence homology at their DNA binding domain (98% identity), and the ERs reveals a high similarity (68%) in the DNA-binding domain and a moderate similarity (36%) in the ligand-binding E domain which may explain the fact that neither ERRα nor ERRγ binds estrogen (Giguere, 2002; Hong, 1999). Structure-function studies showed that ERRs have ligand-binding pockets smaller than those in ERs and these and other studies provided evidence that ERRs may activate gene transcription in a constitutive manner (Greschik et al., 2004; Greschik et al., 2002; Kallen et al., 2004; Nam et al., 2003; Xie et al., 1999). However, it is also worth noting that in silico superimposition of the ligand-binding pocket of ERRα on that of ERα both provided structural support for ERRα and possibly other ERRs being constitutively active, and also revealed a considerable level of sequence identity in the pocket, supporting the hypothesis that structurally similar ligands could be bound by both receptors (Chen et al., 2001). Consistent with this, a variety of recent studies have identified both agonists and antagonists of members of the ERR family, many of which also act as ligands for ERs. Yeast-based assays and mammalian transient transfection assays revealed that two organochlorine pesticides with estrogen-like activity, toxaphene and chlordane, suppress the constitutive activity of ERRα (Yang and Chen, 1999). The synthetic estrogen diethylstilbestrol (DES) and the antiestrogen 4-OH-tamoxifen were also found to be antagonists of ERRs (Coward et al., 2001; Tremblay et al., 2001). Two inverse agonists, thiadiazolopyrimidinone 1a and a derivative XCT790 which interfere with PGC-1/ERRα dependent signaling, have also been reported (Busch et al., 2004; Willy et al., 2004). On the other hand, there are differences in concentrations required and specificities that make identification of a physiological ligand(s) of great interest. In this regard, Suetsugi et al. found that a flavone (6,3′,4′-trihydroxyflavone) and three isoflavone (genistein, daidzein and biochanin A) phytoestrogens can act as agonists of ERRs when used at concentrations not unlike those that activate ERs (Suetsugi et al., 2003). Phenolic Acyl hydrazones have been shown to act as selective agonists for ERRγ (Zuercher et al 2005).

Other data also support the idea that ERRα may impinge on the estrogen pathway. ERRα interacts with ERs through protein-protein interactions in vitro and recognizes the same DNA binding element as ERs (Johnston et al., 1997; Vanacker et al., 1999). ERRα, ERRβ, ERRγ, and ERα can bind to and activate transcription through both the functional estrogen response element (ERE), and the Steroid Factor 1 response element (SFRE). ERβ DNA-binding and transcriptional activity, on the other hand, is restricted to the ERE. ERRα and TRs (Thyroid hormone receptors) can also both bind to and activate transcription through the thyroid response element TRE (Xie et al., 1999). ERRγ has been shown recently to activate transcription via an AP1 site which is also a DNA binding site used by ERs (Huppunen et al., 2004). Several coactivators are known to interact with ERRs and ERs, including steroid receptor coactivator 1 (SRC-1), peroxisome proliferator-activated receptor-gamma coactivator-1 (PGC-1), activator of thyroid and retinoic acid receptor (ACTR) and glucocorticoid receptor interacting protein 1 (GRIP-1) (Xie et al., 1999; Zhang and Teng, 2000). It has been shown that not only ERRα itself (Laganiere et al., 2004; Mootha et al., 2004) but also ERα (Liu et al., 2003) and ERRγ (Zhang and Teng, 2007) binds to and activates transcription of the ERRα promoter. These data suggest possible biological overlap between ERRs and ERs via their DNA binding (ERE, SFRE and AP1) and transcriptional regulatory activity.

Although a growing body of indirect data suggest that ERs and ERRs may be relatively widely distributed, very few studies have addressed directly whether ERRs and ERs are co-expressed in potential target tissues and cells. It has been shown previously that ERRα is expressed and functionally active throughout osteoblast precursor proliferation and differentiation to matrix-synthesizing osteoblasts (Bonnelye et al., 2001) and that ERRα is regulated by estrogen in bone and impinges on the estrogen axis in bone (Bonnelye et al., 2002). Interestingly, it has also been shown by directly and simultaneously assessing the same cells that ERRα is co-expressed in the same cells as the ERs, but that ERs are in fact differentially expressed in different subsets of osteoblasts (Bonnelye and Aubin, 2002), supporting the view that cell-type specific regulation is also a hallmark of these receptor families. Similarly, it has also been shown that ERRα is also expressed in chondrocytes where it regulates chondrogenesis (Bonnelye et al., 2007).

It is clear from human and animal studies that destruction of cartilage occurs in rheumatoid arthritis and other inflammatory arthrides. Available treatments are generally based on administration of anti-inflammatory agents to reduce symptoms and no therapies are available which act at the level of cartilage, to promote restoration of the damaged tissue. Understanding how cartilage and bone are destroyed, and the role of ERRγ and other orphan receptor in said process and other conditions would be beneficial in the diagnosis, treatment and screening assays related to said conditions.

### SUMMARY OF THE INVENTION

The present inventors have shown that ERRγ is highly expressed during chondrogenesis and osteogenesis, regulates the osteoblast-associated gene osteopontin and plays a physiological role in osteoblast differentiation, and regulates a master gene required for chondrocyte development.

In one aspect, the inventors have shown that inhibiting ERRγ through antisense oligonucleotides in osteoblast or chondrocyte cell cultures stimulates bone or cartilage formation respectively.

These findings enable therapeutic intervention to promote bone and/or cartilage formation where this is desirable, for example in conditions involving bone and/or cartilage loss or destruction, by inhibiting ERRγ.

In one aspect, ERRγ plays a physiological role in bone and cartilage formation at both proliferation and differentiation stages.

In one aspect, stimulating ERRγ expression or activity inhibits bone and cartilage formation and antagonising ERRγ expression or activity promotes bone and cartilage formation.

One embodiment of the invention is use of an agent selected from the group consisting of:

(a) an estrogen receptor-related receptor gamma (ERRγ) antagonist;

(b) a purified antibody which binds specifically to ERRγ protein;

(c) an antisense nucleotide sequence complementary to and capable of hybridizing to a nucleotide sequence encoding ERRγ protein; and

(d) an agent which reduces expression of a gene encoding ERRγ protein for the preparation of a medicament for promoting bone and/or cartilage formation in a mammal. In one embodiment the medicament further comprises a pharmaceutically acceptable carrier.

In another embodiment, the invention provides a medicament of the invention for promoting bone and/or cartilage formation that comprises one or more aforementioned agents and optionally a pharmaceutically acceptable carrier. In one embodiment, the medicament increases proliferation of one or more of chondroprogenitor cells, osteoprogenitor cells, chondrocytes and osteocytes.

In another embodiment, a medicament of the invention for promoting bone and/or cartilage formation promotes differentiation of one or more of chondroprogenitor cells, osteoprogenitor cells, chondrocytes and osteocytes.

In another embodiment, a medicament of the invention for promoting bone and/or cartilage formation is used for the treatment of a condition selected from the group consisting of cartilage loss, cartilage degeneration, cartilage injury, bone loss, bone degeneration, and bone injury.

In another embodiment, a medicament of the invention for promoting bone and/or cartilage formation is used for the treatment of arthritis or osteoporosis.

In another embodiment, a medicament of the invention for promoting bone and/or cartilage formation is used for the treatment of a disease selected from the group consisting of ankylosing spondylitis, childhood arthritis, chronic back injury, gout, infectious arthritis, osteoarthritis, osteoporosis, pagets's disease, polymyalgia rheumatica, pseudogout, psoriatic arthritis, reactive arthritis, reiter's syndrome, repetitive stress injury, and rheumatoid arthritis.

In another embodiment, a medicament of the invention for promoting bone and/or cartilage formation is used for systemic or oral administration.

In another embodiment, a medicament of the invention for promoting bone and/or cartilage formation is for intra-articular administration.

In another embodiment, the agent used in the preparation of a medicament of the invention for promoting bone and/or cartilage formation is diethylstilbestrol, 4-hydroxytamoxifen or 4-hydroxytoremifene.

In another embodiment, a medicament of the invention for promoting bone and/or cartilage formation is provided as a solution, tablet, pill or suspension.

In a further embodiment, the invention provides a method for promoting bone and/or cartilage formation in a tissue or cell in vitro comprising contacting the tissue or cell with an agent selected from the group consisting of:

(a) an ERRγ antagonist;

(b) a purified antibody which binds specifically to ERRγ protein;

(c) an antisense nucleotide sequence complementary to and capable of hybridizing to a nucleotide sequence encoding ERRγ protein; and

(d) an agent which reduces expression of the gene encoding ERRγ protein.

In a further embodiment, the tissue in a method of the invention for promoting bone and/or cartilage formation is a cartilage or bone biopsy.

In a further embodiment, the invention provides a use of an agent selected from the group consisting of:
(a) an ERRγ agonist;
(b) a substantially purified ERRγ protein;
(c) a nucleotide sequence encoding ERRγ protein or an effective portion thereof; and
(d) an agent which enhances expression of a gene encoding an ERRγ protein for the preparation of a medicament for inhibiting bone and/or cartilage formation in a mammal.

In a further embodiment, in a medicament of the invention for inhibiting bone and/or cartilage formation in a mammal, said medicament optionally comprises a pharmaceutically acceptable carrier. In another embodiment, the use of the agent is in a medicament of the invention for inhibiting bone and/or cartilage formation in a mammal wherein it reduces proliferation of chondroprogenitor cells, osteoprogenitor cells, chondroblasts, and chondrocytes, and, osteoblasts, and osteocytes.

In a further embodiment, the invention provides a use of an aforementioned agent in a medicament of the invention for inhibiting bone and/or cartilage formation in a mammal that reduces differentiation of chondroprogenitor cells, osteoprogenitor cells, chondroblasts, and chondrocytes, and, osteoblasts, and osteocytes.

In a further embodiment, the invention provides a use of the aforementioned agent in a medicament of the invention for inhibiting bone and/or cartilage formation in a mammal is for the treatment of a condition selected from the group consisting of chondrosarcoma, osteosarcoma, chondrodysplasia and osteodysplasia.

In a further embodiment, said medicament of the invention for inhibiting bone and/or cartilage formation in a mammal is for systemic or oral administration.

In a further embodiment, a medicament of the invention for inhibiting bone and/or cartilage formation in a mammal is for intra-articular administration.

In a further embodiment, an agent in a medicament of the invention for inhibiting bone and/or cartilage formation in a mammal is phenolic acyl hydrazone GSK4716 or GSK9089.

In a further embodiment, a medicament of the invention for inhibiting bone and/or cartilage formation in a mammal is provided as a solution, tablet, pill or suspension.

In a further embodiment, the invention provides a method of promoting bone and/or cartilage formation in a mammal comprising administering to the mammal an effective amount of an agent selected from the group consisting of:
(a) an ERRγ antagonist;
(b) a purified antibody which binds specifically to ERRγ protein;
(c) an antisense nucleotide sequence complementary to and capable of hybridizing to a nucleotide sequence encoding ERRγ protein; and
(d) an agent which reduces expression of the gene encoding ERRγ protein.

In a further embodiment, in a method of the invention for promoting bone and/or cartilage formation in a mammal, the agent of the method increases proliferation of chondroprogenitor cells, osteoprogenitor cells, chondroblasts, and chondrocytes, and, osteoblasts, and osteocytes.

In a further embodiment, in a method of the invention for promoting bone and/or cartilage formation in a mammal, the agent of the method promotes differentiation of chondroprogenitor cells, osteoprogenitor cells, chondroblasts, and chondrocytes, and, osteoblasts, and osteocytes.

In a further embodiment, in a method of the invention for promoting bone and/or cartilage formation in a mammal, the mammal suffers from a condition selected from the group consisting of cartilage loss, cartilage degeneration, cartilage injury, bone loss, bone degeneration and bone injury.

In a further embodiment, in a method of the invention for promoting bone and/or cartilage formation in a mammal, the mammal suffers from arthritis.

In a further embodiment, in a method of the invention for promoting bone and/or cartilage formation in a mammal, the mammal suffers from a disease selected from the group consisting of ankylosing spondylitis, childhood arthritis, chronic back injury, gout, infectious arthritis, osteoarthritis, osteoporosis, pagets's disease, polymyalgia rheumatica, pseudogout, psoriatic arthritis, reactive arthritis, reiter's syndrome, repetitive stress injury, and rheumatoid arthritis.

In a further embodiment, in a method of the invention for promoting bone and/or cartilage formation in a mammal, the agent is administered systemically or orally.

In a further embodiment, in a method of the invention for promoting bone and/or cartilage formation in a mammal, the agent is administered intra-articularly.

In a further embodiment, in a method of the invention for promoting bone and/or cartilage formation in a mammal, the agent is diethylstilbestrol, 4-hydroxytamoxifen or 4-hydroxytoremifene.

In a further embodiment, the invention provides a method of inhibiting bone and/or cartilage formation in a mammal comprising administering to the mammal an effective amount of an agent selected from the group consisting of:
(a) an ERRγ agonist;
(b) a substantially purified ERRγ protein
(c) a nucleotide sequence encoding ERRγ protein; and
(d) an agent which enhances expression of a gene encoding an ERRγ protein.

In a further embodiment, in a method of the invention for inhibiting bone and/or cartilage formation in a mammal, the agent of the method reduces proliferation of chondroprogenitor cells, osteoprogenitor cells, chondroblasts, and chondrocytes, and, osteoblasts, and osteocytes.

In a further embodiment, in a method of the invention for inhibiting bone and/or cartilage formation in a mammal, the agent of the method reduces differentiation of chondroprogenitor cells, osteoprogenitor cells, chondroblasts, and chondrocytes, and, osteoblasts, and osteocytes.

In a further embodiment, in a method of the invention for inhibiting bone and/or cartilage formation in a mammal, the mammal suffers from chondrosarcoma, osteosarcoma, chondrodysplasia or osteodysplasia.

In a further embodiment, in a method of the invention for inhibiting bone and/or cartilage formation in a mammal, the agent is administered systemically or orally.

In a further embodiment, in a method of the invention for inhibiting bone and/or cartilage formation in a mammal, the agent is administered intra-articularly.

In a further embodiment, in a method of the invention for inhibiting bone and/or cartilage formation in a mammal, the agent is phenolic acyl hydrazone GSK4716 or GSK9089.

In further embodiment, the invention provides a method for screening a candidate compound for its ability to modulate ERRγ cartilage and/or bone promoting activity comprising:

(a) providing an assay system for measuring cartilage and/or bone formation; and

(b) measuring the cartilage and/or bone promoting activity of ERRγ in the presence or absence of the candidate compound, wherein a change in ERRγ cartilage and/or bone promoting activity in the presence of the compound relative to ERRγ cartilage and/or bone promoting activity in the absence of the compound indicates an ability to modulate ERRγ cartilage and/or bone promoting activity.

In a further embodiment, in a method of the invention for screening a candidate compound for its ability to modulate ERRγ cartilage and/or bone promoting activity, the change in ERRγ cartilage and/or bone promoting activity in the presence of the compound is an increase in cartilage and/or bone promoting activity, as the case may be.

In a further embodiment, in a method of the invention for screening a candidate compound for its ability to modulate ERRγ cartilage and/or bone promoting activity, the change in ERRγ cartilage and/or bone promoting activity in the presence of the compound is a decrease in cartilage and/or bone promoting activity, as the case may be.

In a further embodiment, the compound identified by a method of the invention for screening a candidate compound for its ability to modulate ERRγ cartilage and/or bone promoting activity is used in the preparation of a medicament for promoting bone and/or cartilage formation in a mammal.

In a further embodiment, the compound identified by a method of the invention for screening a candidate compound for its ability to modulate ERRγ cartilage and/or bone promoting activity is used in the preparation of a medicament for inhibiting bone and/or cartilage formation in a mammal.

Compounds which effect modulation of the bone and/or cartilage promoting activity of ERRγ may be useful to promote bone and/or cartilage formation, if their effect is positive, or to inhibit bone and/or cartilage formation, if their effect is negative.

In accordance with another embodiment of the present invention, a pharmaceutical composition comprises a chondrogenesis and/or osteogenesis promoting amount of an agent selected from the group consisting of:
(a) an ERRγ antagonist;
(b) a purified antibody which binds specifically to ERRγ protein;
(c) an antisense nucleotide sequence complementary to and capable of hybridizing to a nucleotide sequence encoding ERRγ protein; and
(d) an agent which reduces expression of the gene encoding ERRγ protein; and
   a pharmaceutically acceptable carrier.

In accordance with another embodiment of the present invention, a pharmaceutical composition comprises a cartilage and/or bone formation inhibiting amount of an agent selected from the group consisting of:
(a) ERRγ agonist;
(b) a substantially purified ERRγ protein
(c) a nucleotide sequence encoding ERRγ protein; and
(d) an agent which enhances expression of a gene encoding an ERRγ protein; and
   a pharmaceutically acceptable carrier.

In another embodiment, certain compositions of the invention may be used to treat bone and/or joint disease.

In another embodiment, certain compositions of the invention may be used to diagnose bone and/or joint disease.

### SUMMARY OF THE FIGURES

Certain embodiments of the invention are described, reference being made to the accompanying drawings, wherein:

FIG. 1, Panel A is a graph showing the expression level of ERRγ over the proliferation-differentiation time course (in days) of rat calvaria cells in culture.

FIG. 1, Panel B is a graph showing the expression level of ERRγ over the proliferation-differentiation time course (in days) of mouse calvaria cells in culture.

FIG. 2, Panel Aa is a graph showing the expression level of ERRγ mRNA (detected by an antibody against ERRγ prepared by the inventors) throughout the differentiation time course of osteoblasts (rat calvarial cells).

FIG. 2, Panel Ab is a graph showing the expression level of ERRγ mRNA (detected by an antibody against ERRγ prepared by the inventors) throughout the differentiation time course of osteoblasts (rat calvarial cells (different isolate than used in the experiment shown in FIG. 2, Panel Aa)).

FIG. 2, Panel B is a graph showing the expression level of ERRγ mRNA (detected by an antibody against ERRγ prepared by the inventors) throughout the differentiation time course of chondrocytes (C5.18 cells).

FIG. 2, Panel C is a graph showing relative ERRγ expression levels in mouse kidney and brain cells.

FIG. 2, Panel D is a graph showing relative ERRγ expression levels in mouse xyphoid (cartilage), calvarial, and bone cells.

FIG. 3, Panel A is a graph showing the transcriptional regulation by mERRγ1 of mouse osteopontin (OPN) promoter in osteoblastic ROS17/2.8 cells.

FIG. 3, Panel B is a graph showing the transcriptional regulation by mERRγ1 of mouse osteopontin (OPN) promoter in non-osteoblastic HeLa cells.

FIG. 4, Panel A is a graph showing the transcriptional regulation by mERRγ1 of the mSox9 promoter in rat C5.18 chondrocytic cells.

FIG. 4, Panel B is a graph showing the transcriptional regulation by mERRγ1 of the mSox9 promoter in rat C5.18 and HeLa cells.

FIG. 5 is a representation of the ERRγ oligonucleotide treatment time course for regulation studies.

FIG. 6, Panel A is a graph showing bone nodule number in RC cell cultures after treatment with ERRγ antisense oligonucleotides during proliferation.

FIG. 6, Panel B is a graph showing bone nodule number in RC cell cultures after treatment with ERRγ antisense oligonucleotides during differentiation.

FIG. 6, Panel C is a graph (at a different p value from panel B) showing bone nodule number in RC cell cultures after treatment with ERRγ antisense oligonucleotides during differentiation.

FIG. 7 shows cartilage nodule number in C5.18 cell cultures after treatment with ERRγ antisense oligonucleotides during differentiation.

FIG. 8 shows bone and cartilage nodule formation after treatment with ERRγ sense (S), scrambled (Sc), and antisense (AS) oligonucleotides during the differentiation phase.

FIG. 9 shows ERRγ transgene constructs.

FIG. 10 shows a summary table of transgenic lines.

FIG. 11, Panel A is a graph showing preliminary results of bone mineral density in postnatal transgenic female mice (1.5 months).

FIG. 11, Panel B is a graph showing preliminary results of bone mineral density in postnatal transgenic female mice (4 months).

FIG. 11, Panel C is a graph showing preliminary results of bone mineral density in postnatal transgenic male mice (1.5 months).

FIG. 11, Panel D is a graph showing preliminary results of bone mineral density in postnatal transgenic male mice (4.5 months).

FIG. 12, Panel A is a graph showing preliminary results of bone mineral density in postnatal transgenic female mice (5.5/6 months).

FIG. 12, Panel B is a graph showing preliminary results of bone mineral density in postnatal transgenic male mice (6.5 months).

FIG. 13, Panel A is a graph showing preliminary results of bone mineral density in postnatal transgenic male mice (2 months).

FIG. 13, Panel B is a graph showing preliminary results of bone mineral density in postnatal transgenic male mice (4 months).

FIG. 13, Panel C is a graph showing preliminary results of bone mineral density in postnatal transgenic female mice (3.5 months).

FIG. 14 is a protein band showing ERRγ protein expression throughout chondrocyte differentiation sequence in C5.18 cells.

### DETAILED DESCRIPTION OF THE INVENTION

The present inventors have found a new role for the orphan receptor, estrogen receptor-related receptor gamma (ERRγ), in the modulation of bone and cartilage growth and differentiation in mammals.

Bone is a highly metabolically active tissue in which the processes of osteoblastic bone formation and osteoclastic resorption are continuous throughout life. Steroid hormones (e.g., estrogen, progesterone, androgen) play an important role in bone cell development and in maintenance of normal bone architecture (Manolagas et al., 2002; Riggs et al., 2002). A clinically significant manifestation of the loss of estrogen production by the ovary at menopause is the increased bone turnover and accelerated loss of bone mass that leads to increased bone fragility and fracture risk, commonly called osteoporosis (Conference, N.C.D., 1980; Panel, N.C.D., 2001). A positive effect of estrogens on bone homeostasis has been documented in postmenopausal osteoporosis in which bone loss can be stopped by administration of natural or synthetic estrogens (Hodgson et al., 2003). Although the bone-preserving effect of estrogen replacement is indisputable, the molecular and cellular mechanism(s) mediating this effect remain unclear. Indeed, the targeted deletion of one or both ERs fails to recapitulate the severe increase in bone turnover observed after suppression of estrogen after menopause or gonadectomy in female mice (Sims et al., 2002). A number of nuclear steroid receptors are also present in growth plate chondrocytes (van der Eerden et al., 2003). For example, ERs are expressed in cartilage where they are thought to play roles not only in the pubertal growth spurt (Ritzen et al., 2000) but also in cartilage damage associated with osteoarthritis and rheumatoid arthritis both of which predominate in females over males (Richette et al., 2003; Wluka et al., 2000). The coexpression of ERRα with ERα and ERβ in chondrocytes and cartilage has also recently been reported (Bonnelye et al., 2007). Given the homology of ERRγ to the ERs, the evidence that ERRγ interacts with ERα, and that ERRγ regulates ERRα and its own promoter, indicates that ERRγ may play a role in bone and cartilage formation and maintenance.

The inventors show that ERRα, ERRβ, ERRγ, ERα and ERβ are all expressed, but differentially, in osteoblastic and chondrocytic cells in vitro and bone and cartilage in vitro. It has been shown previously that ERRα appears to be more abundantly and widely expressed than either of the two estrogen receptors (Bonnelye and Aubin, 2002). In the case of osteoblasts, the inventors have found that expression of ERRγ mRNA, like that of ERRα, is relatively constant throughout the proliferation-differentiation sequence. Like ERα and ERβ, which are expressed at markedly different levels in bone and cartilage samples, the inventors found that ERRα and ERRγ are also expressed at markedly different levels in these skeletal tissues, with the former at much higher levels than the latter. Taken together with expression level differences in cultured cells, ERRγ, either alone or together with ERs or ERRα, may have widespread roles in both progenitor and more mature cells.

Using promoter-luciferase reporter assays, the inventors found that ERRγ is functional in a bone and cartilage environment to regulate an osteoblast-associated (OPN) and a chondrocyte-associated (Sox9) gene in osteoblasts and chondrocytes, respectively. It has been shown previously that ERRα also regulates Sox9 in chondrocytes (Bonnelye et al., 2007) and OPN in osteoblastic cells (Zirngibl et al., 2006), but ERRγ is a more potent transactivator than ERRα for these genes and promoters. The inventors further found that knocking down ERRγ expression levels with antisense oligonucleotides increases bone nodule formation in rat calvaria osteoblastic cells, suggesting that it enhances osteogenesis (Aubin and Triffitt, 2002). This, together with their expression levels and patterns in bone and cartilage, suggest that ERRα and ERRγ may have both overlapping and unique functions in the skeleton.

Sex steroids, such as estrogen, play a role in the onset and severity of symptoms in arthritis and osteoporosis. Estrogen exerts its activity via its receptors, estrogen receptor α and β, which are members of the nuclear receptor family. There are also members of the family for which ligands have not been identified, the so-called orphan receptors. Amongst these are the estrogen receptor-related receptors ERRα, ERRβ, and ERRγ. Previously, it has been shown that ERRα is highly expressed in bone and cartilage and that it plays a functional role in osteogenesis and chondrogenesis in vitro (Bonnelye et al., 2001; Bonnelye et al., 2007). In addition, a frequent regulatory variant of the ERRα gene is associated with BMD in French-Canadian premenopausal women (Laflamme et al., 2005). However, no skeletal anomalies have been reported in ERRα knockout mice (Luo et al., 2003), raising the possibility that compensatory mechanisms involving other ERR family members may be operative. To begin to address this possibility, the inventors considered whether other ERR family members are expressed in bone and/or cartilage. By real-time PCR, the inventors found not only ERRα, but also ERRβ and ERRγ in adult male long bone (femoral and tibial diaphyses), calvaria, and joint (entire epiphysis including cartilage and secondary ossification zone). ERRβ and ERRγ mRNAs were expressed at levels similar to ERα, which is 8 to 64 fold greater than ERβ, and 50-100 fold less than ERRα in these tissues. ERRγ was also found in cartilage (xyphoid). PCR analysis of rat calvaria (RC) primary osteoblast cultures and the rat chondrogenic cell line C5.18, both of which express high levels of ERRα mRNA and protein, indicated that ERRβ and ERRγ mRNAs are expressed throughout the osteoblast and chondrocyte proliferation-differentiation sequences. Western analysis confirmed the presence of ERRγ protein in chondroblastic and osteoblastic cell lines (not shown).

Immunofluorescence performed on frozen sections of 21dpc rat calvaria also confirmed ERRγ protein in osteoblasts and osteocytes in the parietal bone and in cells at the osteogenic fronts at the sagittal suture. Taken together with the analysis of the capacity of ERRα and ERRγ to regulate cartilage and bone specific promoters (Zirngibl, abstract IBMS, 2007), the data suggest that multiple ERR family members are expressed in the skeleton, where they may play a role in regulation of the development and maintenance of bone and cartilage.

It has been shown previously that ERRα regulates the osteopontin (OPN) promoter through an overlapping AP1/CAAT box element (Zirngibl et al, 2006). To determine whether ERRγ1 regulates OPN in a similar manner, the inventors transfected OPN promoter deletion constructs together with ERRα or ERRγ1 expression plasmids into either HeLa or osteoblastic ROS17/2.8 cells and monitored promoter activity by luciferase assays. To determine which domains of the ERRs were responsible for OPN regulation, various ERR mutations were generated and similarly assayed. Both ERRα and ERRγ1 activate the OPN promoter in HeLa cells. However, whereas ERRα activates via a single site composed of an overlapping AP1/CAAT box, ERRγ1 utilizes a site located in the 5′ untranslated region (UTR) of the OPN promoter in addition to the AP1/CAAT box. In ROS17/2.8 cells, the two ERR isoforms act in opposing ways, with ERRα repressing and ERRγ1 activating transcription. ERRα represses OPN transcription in ROS17/2.8 cells via the same AP1/CAAT box element as used for activation in HeLa cells. ERRγ1, on the other hand, activates the OPN promoter in osteoblastic cells via a distinct site located between the TATA box and the start of transcription. None of the sites that the inventors have identified conform to established ERR response elements (ERREs). Mutations in the activation function 2 (AF2) of ERRα, predicted to abolish activation, surprisingly turn ERRα into a better activator. In contrast, similar AF2 mutations in ERRγ1 abolish activation of the OPN promoter. Mutation of the DNA binding domain of ERRα also abolishes activation or repression in HeLa and ROS17/2.8 cells, respectively. The data indicate, first, that the two ERR isoforms regulate OPN in a cell context dependent manner. Second, the data suggest that although the DNA binding domains of ERRα and ERRγ1 are 93% identical and required for regulation, the receptors bind to distinct OPN promoter elements, suggesting that the two isoforms may co-regulate OPN, and perhaps other genes, without competing for the same site in the promoter. Finally, the results suggest that each isoform interacts differently with co-activators and co-repressors, as highlighted by the AF2 mutation that turn ERRα into a better activator but abolishes activity of ERRγ1.

The invention provides methods and pharmaceutical compositions for promoting bone and/or cartilage formation in a mammal by decreasing ERRγ activity. As used herein, “ERRγ activity” means ERRγ chondrogenic or cartilage inhibiting activity, ie. inhibition of cartilage production, which may occur by inhibition of chondroprogenitor cells and/or chondrocytes and/or inhibition of differentiation of chondroprogenitor cells and/or inhibition of chondrocytes resulting in a decrease of cartilage formation and/or osteogenic or bone inhibiting activity, ie. decrease of bone production.

ERRγ activity may be decreased in a mammal by decreasing the amount of ERRγ protein present or by decreasing the negative chondrogenic effect and/or negative osteogenic effect of existing ERRγ protein. Decreased ERRγ activity may be achieved, for example, by down regulating expression of the ERRγ gene, by gene therapy to provide a nucleotide sequence antisense to that encoding ERRγ protein, by administering an agent which decreases ERRγ expression, by administering a mutated ERRγ protein with decreased or no activity (a dominant-negative) or by administering an ERRγ antagonist. An ERRγ antagonist is a compound which decreases the negative chondrogenic and/or negative osteogenic activity of ERRγ protein.

Agents which increase ERRγ activity may be used for preparation of medicaments for inhibiting cartilage and/or bone formation.

In one embodiment, estrogen analogues, including selective estrogen receptor modifiers (SERMS), may be screened by the methods described herein to select those active as ERRγ antagonists or ERRγ activity down-regulators. For example, synthetic estrogens such as diethylstilbestrol have been found to antagonize ERRγ. Furthermore, the selective estrogen receptor modulators 4-hydroxytamoxifen and 4-hydroxytoremifene antagonize ERRγ.

The cartilage formation promoting methods and compositions of the invention can be employed to treat conditions associated with cartilage loss, cartilage degeneration or cartilage injury. Such conditions include the various disorders described collectively as arthritis. The bone formation promoting methods and compositions of the invention can be employed to treat conditions associated with bone loss, such as in osteoporosis, bone degeneration or bone injury. ERRγ expression in the vagina of juvenile female mice has been shown to be regulated by isoflavone (Takashima-Sasaki, et al., 2007).

Arthritis is a term used to designate generally diseases of the joint. Arthritis includes many different conditions but is characterized generally by the presence of joint inflammation. Inflammation is involved in many forms of arthritis and results, among other things, in the destruction of joint cartilage.

The list of diseases that are included in the term arthritis includes, but is not limited to, ankylosing spondylitis, childhood arthritis, chronic back injury, gout, infectious arthritis, osteoarthritis, polymyalgia rheumatica, pseudogout, psoriatic arthritis, reactive arthritis, reiter's syndrome, repetitive stress injury, and rheumatoid arthritis.

Cartilage destruction or injury can also result from joint surgery, joint injury and obesity.

A number of symptomatic treatments for arthritis exist, including analgesics and non-steroidal anti-inflammatory agents. Other treatments for inflammatory arthritis include disease modifying agents (DMARDS) such as gold salts, methotrexate, sulfasalazine, hydroxychloroquine, chloroquine and azathioprine. Steroids and corticosteroids are anti-inflammatory agents that are used to treat the inflammation underlying cartilage destruction.

No current arthritis therapy acts at the level of cartilage. Although many of the treatments for arthritis may be able to reduce the effects of the inflammation which causes cartilage destruction, these treatments do not promote cartilage regrowth in the affected tissue.

The present invention provides methods and pharmaceutical compositions for treating arthritis by decreasing ERRγ activity. ERRγ activity may be decreased as described above.

An ERRγ antagonist or an agent which inhibits ERRγ expression may be administered systemically to the subject in need of treatment, or may be administered locally, for example by intra-articular injection.

An antisense sequence such as an antisense oligonucleotide or an antisense adenovirus can be administered by gene therapy as described above, preferably by local injection. Antibodies or antagonists can be administered locally, or systemically if target specific.

If ERRγ activity is to be decreased by gene therapy, a preferred method is by administration of a suitable vector, such as an adenovirus or an adeno-associated virus carrying a sequence antisense to the ERRγ gene, by intra-articular injection. Such intra-articular gene administration has been described by Goater et al., (2000) and van Lent et al. (2002).

Another group of diseases involves unwanted or inappropriate cartilage formation. Such diseases include chondrosarcomas and chondrodysplasias. The present invention provides methods and pharmaceutical compositions for inhibiting bone and/or cartilage formation by increasing ERRγ activity and thereby treating such disorders. ERRγ activity may be increased by increasing the amount of ERRγ protein being produced or by enhancing the activity of ERRγ protein. This may be achieved, for example, by administering a nucleotide sequence as described herein, or an agent which enhances ERRγ expression, a substantially purified ERRγ protein or an ERRγ agonist. An ERRγ agonist is a compound which increases the negative chondrogenic and/or negative osteogenic activity of ERRγ protein. For example, phenolic acyl hydrazones GSK4716 and GSK9089 are agonists of ERRγ.

In a further embodiment, the invention provides a method for assessing the ERRγ level or activity of a tissue, which can be used as a screening method for possible susceptibility to cartilage degeneration or as a method for monitoring treatment efficacy during treatment of a cartilage degenerative disorder. For example, subjects such as athletes or the overweight, who are at increased risk of osteoarthritis, could be screened for higher than normal cartilage ERRγ, which would suggest susceptibility to development of osteoarthritis. Subjects being treated for rheumatoid arthritis could have their cartilage ERRγ level monitored at intervals to assess whether normal ERRγ levels were being restored or maintained. ERRγ levels can be measured in samples of biopsied joint cartilage tissue, for example by RT-PCR of mRNA as described herein and in Bonnelye et al., (2001) or, less quantitatively, by immunolabelling techniques such as those described in Bonnelye et al., (2001).

The invention also provides a method for screening a candidate compound for its ability to modulate ERRγ chondrogenic and/or osteogenic activity in a suitable system, by examining ERRγ chondrogenic and/or osteogenic activity in the presence or absence of the candidate compound. A change in ERRγ chondrogenic and/or osteogenic activity in the presence of the compound relative to ERRγ chondrogenic and/or osteogenic activity in the absence of the compound indicates that the compound modulates ERRγ chondrogenic and/or osteogenic activity. If ERRγ chondrogenic inhibiting and/or osteogenic inhibiting activity is increased relative to the control in the presence of the compound, the compound is potentially useful as an inhibitor of chondrogenesis and/or osteogenesis, as the case may be. By means of the assays described herein, one of skill in the art can readily determine whether such a compound caused increased ERRγ expression or acted as an ERRγ agonist, to increase activity of ERRγ protein. Conversely, if ERRγ chondrogenic inhibiting and/or osteogenic inhibiting activity is decreased in the presence of the compound, relative to the control, the compound is potentially useful as a promoter of chondrogenesis and/or osteogenesis, as the case may be. It can be determined by means of the assays described herein whether such a compound caused decreased ERRγ expression or acted as an ERRγ antagonist, to decrease activity of ERRγ protein.

Any assay system which enables one to measure the chondrogenic activity or cartilage promoting activity of ERRγ may be employed as the basis of the screening method. Suitable assay systems include, for example, measurement of chondroprogenitor proliferation, cartilage nodule formation or increase of chondroblast markers stimulated by decreased ERRγ expression in a chondrogenic cell line such as C5.18, as described herein.

An example of an assay system that might enable one to measure osteogenic activity or bone promoting activity of ERRγ is described herein and in Bonnelye et al., 2001, and includes osteoprogenitor proliferation, bone nodule formation, or increase in osteoblast markers stimulated by decreased ERRγ expression, although it will be appreciated that other assay systems might also enable one to measure osteogenic activity or bone promoting activity of ERRγ.

Candidate compounds may be subjected to an initial screening for their effect on activation of the ERRγ promoter, before proceeding to the more involved testing of their biological effect in the screening method described above. While ERRs do not respond to natural estrogens, these receptors recognise the estrogen response element and have been shown to activate and repress gene expression in the absence of endogenously added ligand. One of skill in the art can refer to Shi et al. (1997), Yang et al. (1999) and Tremblay et al. (2001) for suitable methods.

In accordance with a further embodiment of the invention, the ERRγ signalling pathway may be modulated by modulating the binding of the ERRγ to an ERRγ binding partner. Such a binding partner may include for example the orphan nuclear receptor small heterodimer partner (SHP). ERRγ can be used to upregulate the transcription and thus expression of genes which work together with ERRγ to affect cartilage development.

The invention further provides methods for screening candidate compounds to identify those able to modulate signaling by ERRγ through a pathway involving ERRγ.

For example, the invention provides screening methods for compounds able to bind to ERRγ which are therefore candidates for modifying the chondrogenic and/or osteogenic activity of ERRγ. Various suitable screening methods are known to those in the art (for example, Hong et al., (1999), Gaillard et al., (2006)), including immobilization of ERRγ on a substrate and exposure of the bound ERRγ to candidate compounds, followed by elution of compounds which have bound to the ERRγ.

Co-immunoprecipitation of protein binding partners with an ERRγ-specific antibody will allow the identification of cartilage-specific or bone-specific binding partners which contribute to ERRγ chondrogenic inhibiting and osteogenic inhibiting activity, respectively.

The invention also provides a method of modulating a ERRγ signaling pathway by increasing or decreasing the availability of ERRγ or by modulating the function of the ERRγ.

The invention further provides methods for preventing or treating diseases characterised by an abnormality in an ERRγ signaling pathway which involves ERRγ, by modulating signaling in the pathway.

According to another aspect of the present invention is a method for suppressing in a mammal, the proliferation of a chondrocytic and/or osteocytic cell capable of being stimulated to proliferate by upregulating ERRγ, the method comprising administering to the mammal an effective amount of an ERRγ agonist or a substantially purified ERRγ protein.

The invention also enables transgenic non-human animal models, which may be used for study of the effects on chondrogenesis and/or osteogenesis of over and under expression of the ERRγ gene, for the screening of candidate compounds as potential agonists or antagonists of this receptor and for the evaluation of potential therapeutic interventions.

The transgenic animals of the invention may also provide models of disease conditions associated with abnormalities of ERRγ expression. Animal species suitable for use in the animal models of the invention include mice, rats, rabbits, dogs, cats, goats, sheep, pigs and non-human primates.

Animal models may be produced which over-express ERRγ by inserting a nucleic acid sequence encoding ERRγ into a germ line cell or a stem cell under control of suitable promoters, using conventional techniques such as oocyte or blastocyst microinjection or transfection or microinjection into stem cells. A cartilage specific promoter such as the Type II collagen promoter may be used, for example. Furthermore, a bone specific promoter such as COL1A1 or osteocalcin may be used, for example. Animal models can also be produced by homologous recombination to create artificially mutant sequences (knock-in targeting of the ERRγ gene) or loss of function mutations (knock-out targeting of the ERRγ gene). For example, knock-out animal models can be made using the tet-receptor system described U.S. Pat. No. 5,654,168 or the Cre-Lox system described, for example, in U.S. Pat. Nos. 4,959,717 and 5,801,030.

In accordance with one embodiment of the invention, transgenic animals are generated by the introduction of an ERRγ transgene into a fertilized animal oocyte, with subsequent growth of the embryo to birth as a live animal. The ERRγ transgene is a transcription unit which directs the expression of ERRγ gene in eukaryotic cells. To create the transgene, ERRγ gene is ligated with an eukaryotic expression module. The basic eukaryotic expression module contains a promoter element to mediate transcription of ERRγ sequences and signals required for efficient termination and polyadenylation of the transcript. Additional elements of the module may include enhancers which stimulate transcription of ERRγ sequences. The most frequently utilized termination and polyadenylation signals are those derived from SV40. The choice of promoter and enhancer elements to be incorporated into the ERRγ transgene is determined by the cell types in which ERRγ gene is to be expressed. To achieve expression in a broad range of cells, promoter and enhancer elements derived from viruses may be utilized, such as the herpes simplex virus thymidine kinase promoter and polyoma enhancer. To achieve exclusive expression in a particular cell type, specific promoter and enhancer elements could be used, such as the promoter of the mb-1 gene and the intronic enhancer of the immunoglobulin heavy chain gene. In a preferred embodiment, a cartilage specific promoter such as the promoter of Type II collagen may be used to target expression in chondrocytes (Bridgewater 1998; Lefebvre 1996). In one embodiment, the bone specific promoter is COL1A1 or osteocalcin.

The ERRγ transgene is inserted into a plasmid vector, such as pBR322 for amplification. The entire ERRγ transgene is then released from the plasmid by enzyme digestion, purified and injected into an oocyte. The oocyte is subsequently implanted into a pseudopregnant female animal. Southern blot analysis or other approaches are used to determine the genotype of the founder animals and animals generated in the subsequent backcross and intercross.

Such ERRγ-deficient mice will provide a model for study of the role of ERRγ in chondrocyte and/or osteocyte differentiation and proliferation and general skeletal development. Such animals will also provide tools for screening candidate compounds for their interaction with ERRγ or the signalling pathway activated by ERRγ.

The invention also provides pharmaceutical compositions for inhibiting cartilage and/or bone formation, comprising as active ingredient a substantially purified ERRγ protein, an ERRγ agonist or an isolated nucleotide sequence encoding ERRγ protein.

ERRγ protein may be produced by conventional recombinant techniques permitting expression of ERRγ by a suitable host cell. A DNA encoding ERRγ may be prepared as described, for example, in Zirngibl et al., (2006).

Techniques for production of proteins by recombinant expression are well known to those in the art and are described, for example, in Sambrook et al. (1989) or latest edition thereof. Suitable host cells include E. coli or other bacterial cells, yeast, fungi, insect cells or mammalian cells.

The invention provides for compositions for promoting cartilage and/or bone formation comprising as active ingredient an ERRγ antagonist obtained by using a screening method as described herein.

A nucleotide sequence encoding ERRγ protein may be administered to a subject either in vivo or ex vivo. Expression may be targeted to a selected cell or tissue by use of an appropriate promoter.

The invention also provides pharmaceutical compositions for increasing or promoting cartilage and/or bone formation, comprising as active ingredient an antibody which binds specifically to ERRγ, an ERRγ antagonist or a negative regulator such as an antisense nucleic acid or a dominant negative mutant version of the ERRγ gene.

The invention provides for compositions for reducing cartilage and/or bone formation comprising as active ingredient an ERRγ agonist obtained by using a screening method as described herein.

Antibodies which bind specifically to ERRγ protein may be made by conventional techniques.

The term “antibodies” includes polyclonal antibodies, monoclonal antibodies, single chain antibodies and fragments such as Fab fragments.

In order to prepare polyclonal antibodies, fusion proteins containing defined portions or all of an ERRγ protein can be synthesized in bacteria by expression of the corresponding DNA sequences, as described above. Fusion proteins are commonly used as a source of antigen for producing antibodies. Alternatively, the protein may be isolated and purified from the recombinant expression culture and used as source of antigen. Either the entire protein or fragments thereof can be used as a source of antigen to produce antibodies.

The purified protein is mixed with Freund's adjuvant and injected into rabbits or other appropriate laboratory animals. Following booster injections at weekly intervals, the animals are then bled and the serum isolated. The serum may be used directly or purified by various methods including affinity chromatography to give polyclonal antibodies.

Monoclonal anti-ERRγ antibodies may be produced by methods well known in the art. Briefly, the purified protein or fragment thereof is injected in Freund's adjuvant into mice over a suitable period of time, spleen cells are harvested and these are fused with a permanently growing myeloma partner and the resultant hybridomas are screened to identify cells producing the desired antibody. Suitable methods for antibody preparation may be found in standard texts such as Barreback, E. D. (1995).

The pharmaceutical compositions of the invention may comprise, in addition to the active ingredient, one or more pharmaceutically acceptable carriers.

Administration of an effective amount of a pharmaceutical composition of the present invention means an amount effective, at dosages and for periods of time necessary to achieve the desired result. This may also vary according to factors such as the disease state, age, sex, and weight of the subject, and the ability of the composition to elicit a desired response in the subject. Dosage regima may be adjusted to provide the optimum therapeutic response. For example, several divided doses may be administered daily or the dose may be proportionally reduced as indicated by the exigencies of the therapeutic situation.

By pharmaceutically acceptable carrier as used herein is meant one or more compatible solid or liquid delivery systems. Some examples of pharmaceutically acceptable carriers are sugars, starches, cellulose and its derivatives, powdered tragacanth, malt, gelatin, collagen, talc, stearic acids, magnesium stearate, calcium sulfate, vegetable oils, polyols, agar, alginic acids, pyrogen-free water, isotonic saline, phosphate buffer, and other suitable non-toxic substances used in pharmaceutical formulations. Other excipients such as wetting agents and lubricants, tableting agents, stabilizers, anti-oxidants and preservatives are also contemplated.

The compositions described herein can be prepared by known methods for the preparation of pharmaceutically acceptable compositions which can be administered to subjects, such that an effective quantity of the active substance is combined in a mixture with a pharmaceutically acceptable carrier. Suitable carriers and formulations adapted for particular modes of administration are described, for example, in Remington's Pharmaceutical Sciences (Remington's Pharmaceutical Sciences, Mack Publishing Company, Easton, Pa., USA 1985). On this basis the compositions include, albeit not exclusively, solutions of the substance in association with one or more pharmaceutically acceptable vehicles or diluents, and contained in buffered solutions with a suitable pH and iso-osmotic with the physiological fluids.

The pharmaceutical compositions of the invention may be administered therapeutically by various routes such as by injection or by oral, nasal, intra-articular, intra-vertebral, buccal, rectal, vaginal, transdermal or ocular administration in a variety of formulations, as is known to those skilled in the art.

The present invention enables also a screening method for compounds of therapeutic utility as agonists of the chondrogenic inhibiting and/or osteogenic inhibiting activity of ERRγ. Such agonist compounds are useful, for example, to reduce or prevent differentiation and maturation of chondrocytes and/or osteocytes. ERRγ agonists may also be used in the treatment of cartilage related disorders involving inappropriate cartilage growth and/or bone related disorders involving inappropriate bone growth. Those skilled in the art will be able to devise a number of possible screening methods for screening candidate compounds for ERRγ agonism.

A screening method may also be based on binding to the ERRγ receptor. Such competitive binding assays are well known to those skilled in the art. Once binding has been established for a particular compound, a biological activity assay is employed to determine agonist or antagonist potential.

### EXAMPLES

The examples are described for the purposes of illustration and are not intended to limit the scope of the invention.

Methods of biochemistry, molecular biology, histology and immunology referred to but not explicitly described in this disclosure and examples are reported in the scientific literature and are well known to those skilled in the art.

### Culturing of Mouse and Rat Calvaria Cells

Cells were isolated from 21-day-old fetal Wistar rat or neonatal mouse calvariae or were the chondrogenic C5.18 cell line and were grown for osteogenic or chondrogenic differentiation as described previously (Bonnelye et al., 2001; Bonnelye et al., 2007). Medium was replaced every second day and cells were cultured at 37° C. in a 5% CO₂ humidified atmosphere for 15-20 days until mineralized bone nodules or cartilage nodules had formed in control cultures.

### Real-Time PCR

Total RNA was extracted with Trizol reagent from tissues and cells according to the manufacturer's directions and previously published protocols (Bonnelye et al., 2002).

Samples of total RNA (1.5-5 μg) were reverse transcribed and semiquantitative real-time RT-PCR was carried out by using the LightCycler system (SYBR Green; Applied Biosystems, Foster City, Calif.) according to the manufacturer's instructions. Amplimers were quantified in triplicate samples for each gene and normalized to corresponding L32 values. Primer concentrations were 0.2 uM (ERRS) or 0.5 uM (ERs), and PCR was done with 95° C. for 10 min; 95° C. for 15 secs, 59° C. for 1 min, 40-50 cycles. Primers used were as follows:

| | |
|---|---|
| L32 | |
| Rat | |
| CACAATGTCAAGGAGCTGGAAGT-forward | (SEQ ID NO: 1) |
| TCTACGATGGCTTTTCGGTTCT-reverse | (SEQ ID NO: 2) |
| Mouse | |
| CACAATGTCAAGGAGCTGGAAGT-forward | (SEQ ID NO: 3) |
| TCTACAATGGCTTTTCGGTTCT-reverse | (SEQ ID NO: 4) |
| Human | |
| CAAGGAGCTGGAAGTGCTGC-forward | (SEQ ID NO: 5) |
| CAGCTCTTTCCACGATGCCT-reverse | (SEQ ID NO: 6) |
| ERRα | |
| Rat | |
| CCTGCAAAGCCTTCTTCAAGA-forward | (SEQ ID NO: 7) |
| GCGTCTCCGCTTGGTGAT-reverse | (SEQ ID NO: 8) |
| Mouse | |
| TCGAGAGATAGTGGTCACCATCAG-forward | (SEQ ID NO: 9) |
| CTTCCATCCACACACTCTGCAG-reverse | (SEQ ID NO: 10) |
| Human | |
| ACCGAGAGATTGTGGTCACCA-forward | (SEQ ID NO: 11) |
| CATCCACACGCTCTGCAGTACT-reverse | (SEQ ID NO: 12) |
| ERRβ | |
| Mouse and Rat | |
| TGAGATCACCAAACGGAGGC-forward | (SEQ ID NO: 13) |
| GAACTCGGTCAAGGCGCA-reverse | (SEQ ID NO: 14) |
| Human | |
| GTTTCCTGAGGTCAAGGACTTCC-forward | (SEQ ID NO: 15) |
| AGCCTGTGTGACCTGCAGC-reverse | (SEQ ID NO: 16) |
| ERRγ | |
| Rat | |
| GACATCGCCTCTGGGTATCAC-forward | (SEQ ID NO: 17) |
| GCCGGGCAGCTGTACTCTAT-reverse | (SEQ ID NO: 18) |
| Mouse | |
| TGTGACTTGGCTGACCGAGA-forward | (SEQ ID NO: 19) |
| TGGAGGAGGCTCATCTGGTCT-reverse | (SEQ ID NO: 20) |
| Human | |
| ACAAAGCGCAGACGTAAATCC-forward | (SEQ ID NO: 21) |
| CGACCTCCACGTACTCTGTCA-reverse | (SEQ ID NO: 22) |
| ERα | |
| Mouse and Rat | |
| GGCTGCGCAAGTGTTACGAA-forward | (SEQ ID NO: 23) |
| CATTTCGGCCTTCCAAGTCAT-reverse | (SEQ ID NO: 24) |
| Human | |
| AAGAGGGTGCCAGGCTTTG-forward | (SEQ ID NO: 25) |
| TGGAGCGCCAGACGAGA-reverse | (SEQ ID NO: 26) |
| ERβ | |
| Rat | |
| TTGGTGTGAAGCAAGATCACTAGAG-forward | (SEQ ID NO: 27) |
| GACTAGTAACAGGGCTGGCACAA-reverse | (SEQ ID NO: 28) |
| Mouse | |
| TTGGTGTGAAGCAAGATCACTAGAA-forward | (SEQ ID NO: 29) |
| GACTAGTAACAGGGCTGGCACAA-reverse | (SEQ ID NO: 30) |
| Human | |
| CCAACACCTGGGCACCTTT-forward | (SEQ ID NO: 31) |
| TCTAGCGATCTTGCTTCACACC-reverse | (SEQ ID NO: 32) |

### Antisense and Sense Oligonucleotide Treatments

Rat calvarial cells were plated into 24 well plates at 10⁴ cells/well. Antisense oligonucleotide inhibition of ERRγ expression was accomplished with a 20-base phosphorothioate-modified oligonucleotide. Control dishes were treated with the complementary sense oligonucleotide or no oligonucleotide. Briefly, oligonucleotide concentrations that have been found previously not to be toxic (0.5 μM to 2 μM) were added directly to cells during the proliferation (day 1-5) or differentiation phase (day 5 (end of proliferation) to 11) in standard medium as above supplemented with 50 μg/ml ascorbic acid, 10 mM sodium β-glycerophosphate. Medium was changed every 2 days and fresh oligonucleotides were added at each change. At day 15, cultures were terminated and stained for alkaline phosphatase according to previous protocols (Bonnelye et al., 2001). Oligonucleotide sequences were:

| | |
|---|---|
| rERRγ AS: ggtGGTTGACGCTGTCCGTCagg | (SEQ ID NO: 33) |
| rERRγ S: cctGACGGACAGCGTCAACCacc | (SEQ ID NO: 34) |
| rERRγ Sc: cagGTGCTCGGTACGGTGGCtgt | (SEQ ID NO: 35) |

### Transfections and Transactivation Assay

Cells were transfected with full length mouse ERRγ (cloned into a modified pcDNA3.1-vector) (Invitrogen) using Lipofectamine 2000 (Invitrogen) following manufacturer's instructions. Each transfection contained empty vector (control) or ERRγWT or mutant expression plasmid, pGL3 mSox9 (−1850 to +107) promoter vector (generous gift of T. M. Underhill, University of British Columbia, Calif.) or pGL3 mOPN (−1981+78) promoter vector (generous gift of D. A. Towler, Washington School of Medicine, USA) and their respective promoter deletions with the pRL tk plasmid (Promega) for internal control. Luciferase activities were measured on an EG&G Berthold Microplate Luminometer LB96V (EG&G Berthold GMBH&Co. Germany) using the Dual-Luciferase reporter assay system (Promega). The experiments were done in triplicate and repeated at least three times.

### Preparation of Transgenic Mice

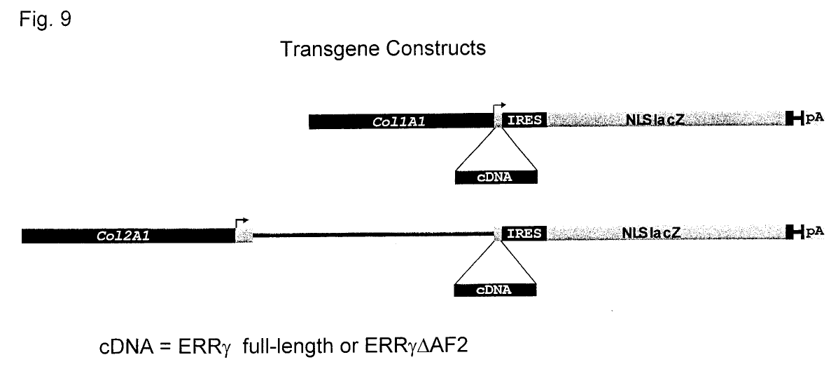

A 2.3 kb fragment of the mouse Col1a1 promoter (Rossert et al, 1995) or a 6.0 kb fragment (Zhou et al, 1995) of the mouse Col2a1 promoter was cloned upstream of ERRγ full-length or ERRγ with amino acids 430-457 deleted (ERRγΔAF2 or ERRγmAF2 or ERRγΔ430-457 and β-galactosidase downstream (FIG. 9)). The constructs were tested in vitro by transient transfections in ROS17/2.8 osteosarcoma cells and C5.18 chondrogenic cells respectively. DNA was microinjected into FVB or CD1 mouse oocytes to generate transgenic animals using standard approaches (Nagy et al, 2002). Founder lines tested positive by PCR analysis for the presence of the transgene. Transgenic founders were bred to CD1 mice and transgenic and wild type progeny were tested for measurements of bone mineral density by PIXImus.

### Example 1

### Expression Studies

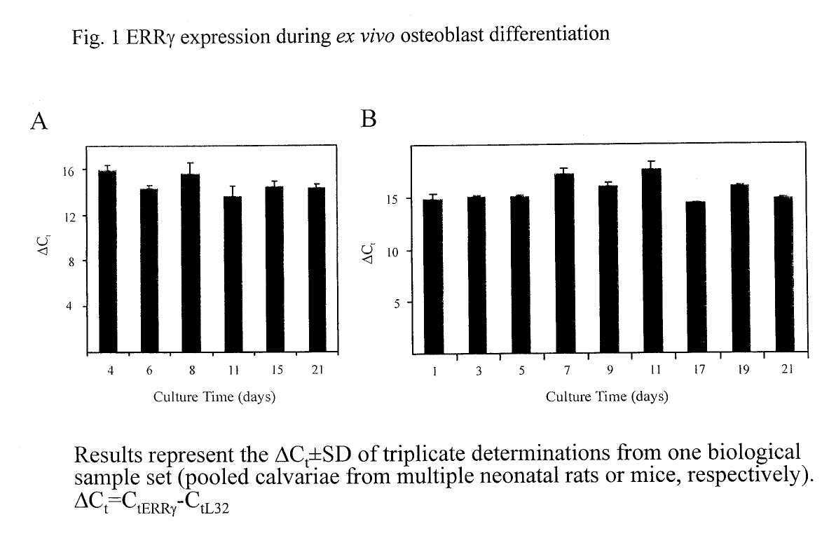

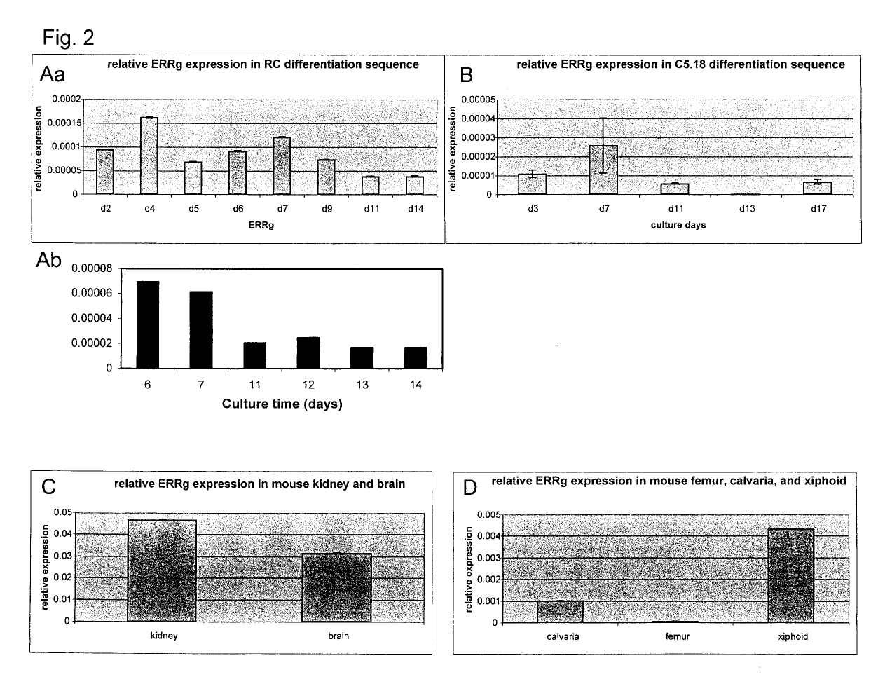

Expression of ERRγ mRNA in such metabolically active tissues as skeletal muscle, heart and kidney was confirmed (Zhang and Teng, 2007) and, for the first time, its expression in cortical bone is reported (femur) (Table 1, below), mouse (MC3T3-E1) and rat (ROS17/2.8) osteoblastic cell lines, and primary cultures of mouse and rat calvaria cells (Table 2, below). In the case of the latter, the inventors found that ERRγ mRNA is expressed at relatively constant levels throughout the proliferation-differentiation sequence from osteoprogenitor to mature osteoblast stages (FIG. 1). In FIG. 1, semi-quantitative real-time PCR was used to assess the expression level of ERRγ over the proliferation-differentiation time course (in days) of rat (A) and mouse (B) calvaria cells in culture. Further studies have shown that ERRγ mRNA expression tends to be highest during proliferation phase in both osteoblast and chondroblast cells (FIG. 2). The inventors also found expression of ERRγ in cartilage (mouse epiphyseal cartilage) and a chondrocyte cell line (C5.18 cells) (Table 2) (FIG. 2). Levels of expression in bone and cartilage tissue and cell samples are lower than ERRα but usually comparable to or even higher than the ERs which are differentially expressed in the samples tested (Table 1). For example, ERRγ and ERα are expressed at levels considerably lower than ERRα but considerably higher than ERβ in rat femur. On the other hand, in cartilage, ERRα and ERα are expressed at similar levels that are markedly higher than the similar levels of ERRγ and ERβ (Table 1). All of the known ERR family members, and known ER family members are co-expressed in the tissues analyzed, including bone and cartilage. ERRα, ERRβ and ERRγ are expressed in osteoblast and chondrocyte cell lines, and bone and cartilage tissue. ERRγ expression level in skeletal tissues is lower than seen in known positive tissues but similar to that of the ERs, ERα and ERβ, and ERRβ, all of which are several fold lower than ERRα.

ERRγ mRNA and protein (as detected by an antibody against ERRγ) are expressed throughout the differentiation time course of osteoblasts (rat calvarial cells; Aa and Ab of FIG. 2) and chondrocytes (C5.18 cells; B of FIG. 2). FIG. 2, panels Aa, Ab, and B show ERRγ mRNA results only, but the inventors have also found that ERRγ protein is expressed throughout the differentiation time course.

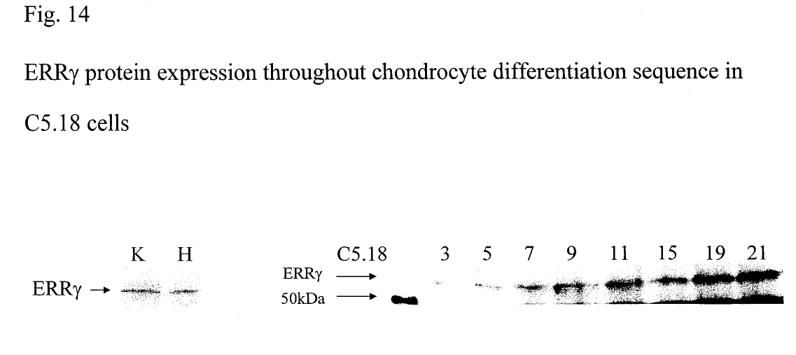

FIG. 14 shows the inventors results where a peptide (LNPQLVQPAKKPYNK)-specific ERRγ antibody was generated. ERRγ protein was detected in mouse kidney (K) and heart (H), as well as throughout the differentiation sequence in rat C5.18 cells. In other experiments, the inventors have evidence that the antibody also detects ERRγ in rat calvaria cells.

ERRγ mRNA expression tends to be highest during the proliferation phase in both osteoblast and chondroblast cells. ERRγ expression level in skeletal tissues is lower than seen in known positive tissues (kidney, brain; C of FIG. 2). ERRγ expression is higher in cartilage (xyphoid) than in calvarial and femoral bone (D of FIG. 2).

### Example 2

### Regulation Studies

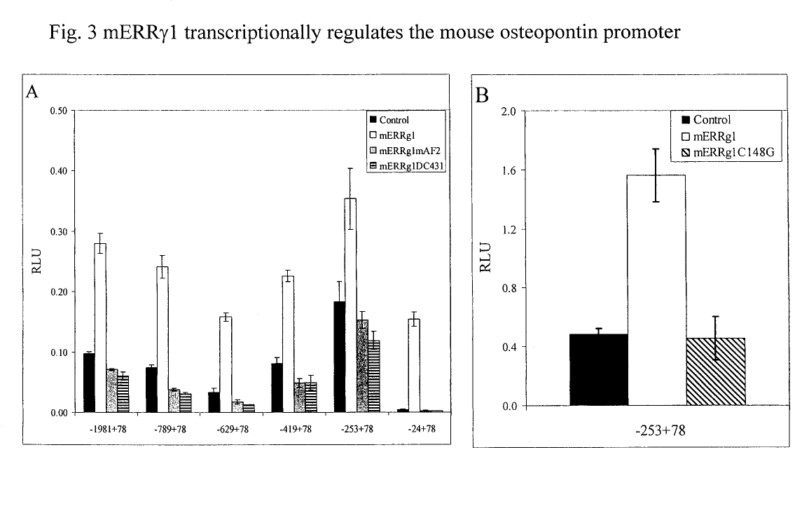

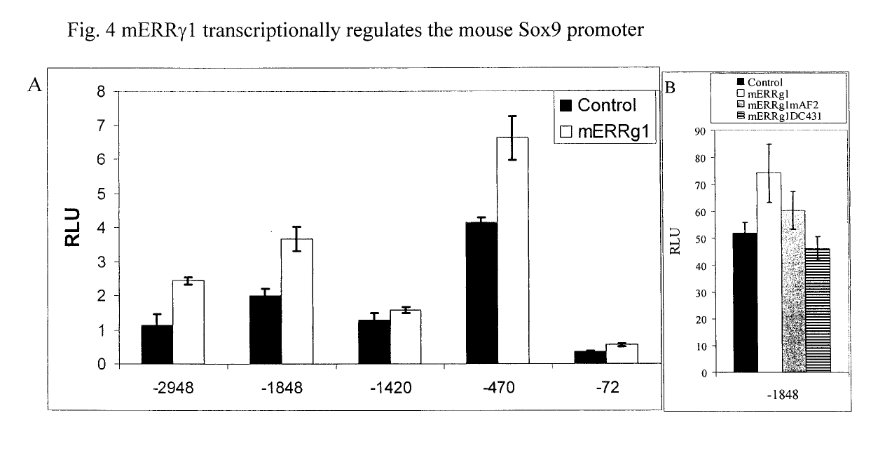

To address whether ERRγ functionally regulates bone (osteopontin; OPN) and/or cartilage (Sox9) genes, promoter-luciferase reporter assays were performed as per Bonnelye et al., 2007 and Zirngibl et al., 2006. As seen in FIG. 3, mERRγ1 activates the mouse OPN promoter in osteoblastic ROS17/2.8 (A) and non-osteoblastic human HeLa (B) cells. Progressive 5′ deletions of the OPN promoter are activated by wild type mERRγ1, but not by C-terminal AF2 transactivation (mERRγ1 mAF2, mERRγ1DC431) or DNA binding (mERRγ1C148G) mutants (FIG. 3). Similarly, as seen in FIG. 4, mERRγ1 activates the mSox9 promoter in rat C5.18 chondrocytic (A) and HeLa (B) cells. Progressive 5′ mSox9 promoter deletions are activated by mERRγ1 (A, but C-terminal AF2 transactivation domain deletions of mERRγ1 lose the ability to activate the mSox9 promoter (B) (FIG. 4).

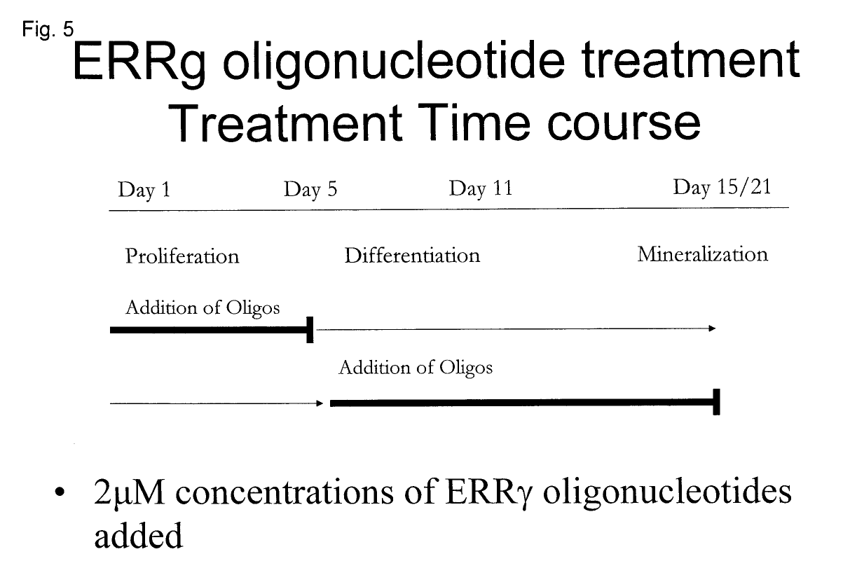

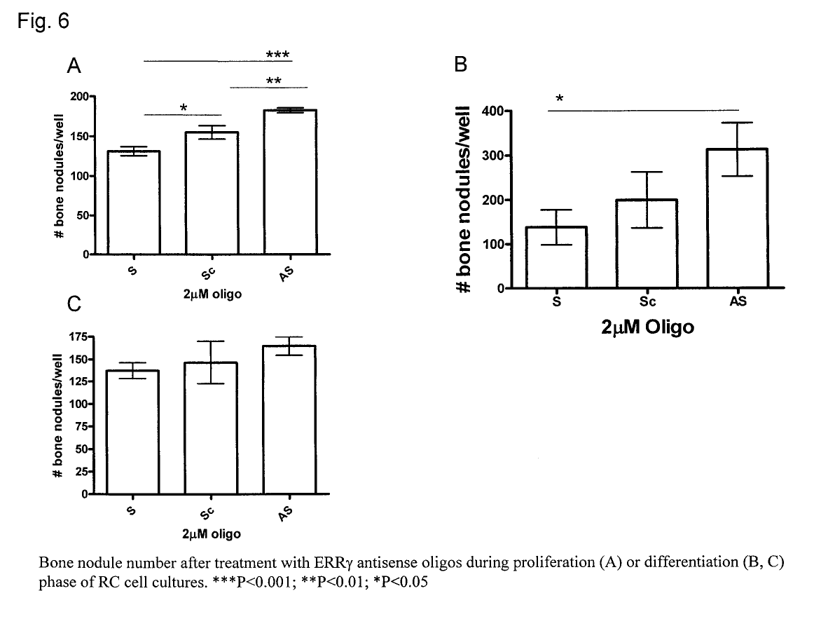

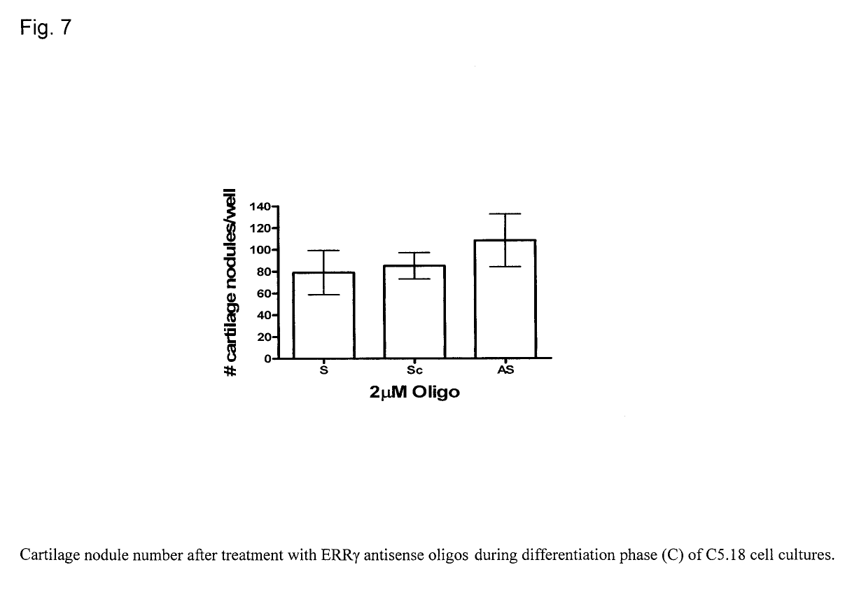

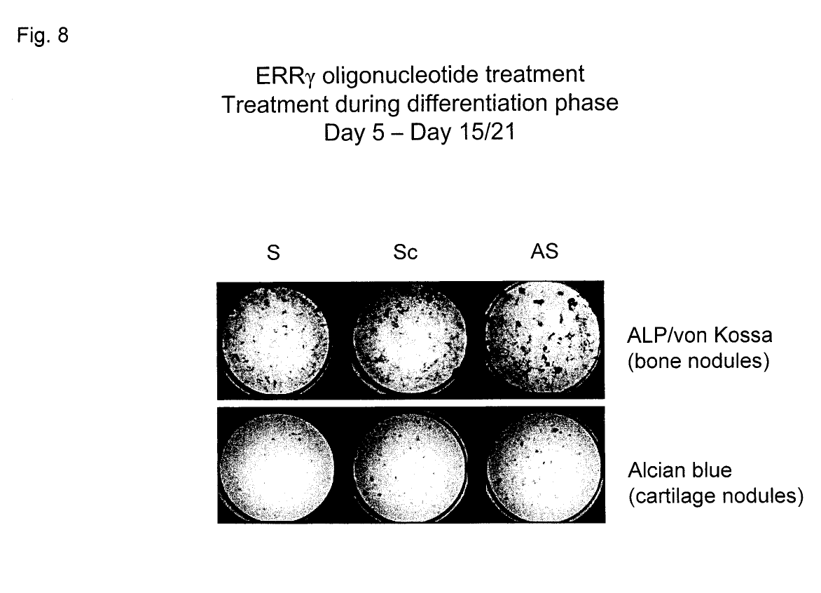

Further studies of ERRγ oligonucleotide treatment along a time course (FIG. 5) were done to determine bone and cartilage nodule number (FIGS. 6, 7 and 8). This antisense oligonucleotide treatment of ERRγ significantly increased bone nodule formation in RC cultures during the proliferation and differentiation phases and tended to increase cartilage nodule formation in C5.18 cultures treated during the differentiation phase suggesting that, in contrast to ERRα, ERRγ is a negative regulator of osteogenesis and chondrogenesis.

### Example 3

### Knock Down Studies

To address further a functional role for ERRγ in bone cells, expression of ERRγ was knocked down in rat calvaria cells using antisense oligonucleotide treatments.

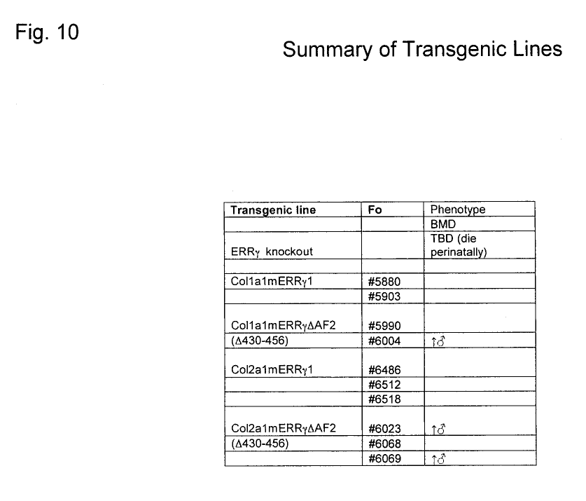

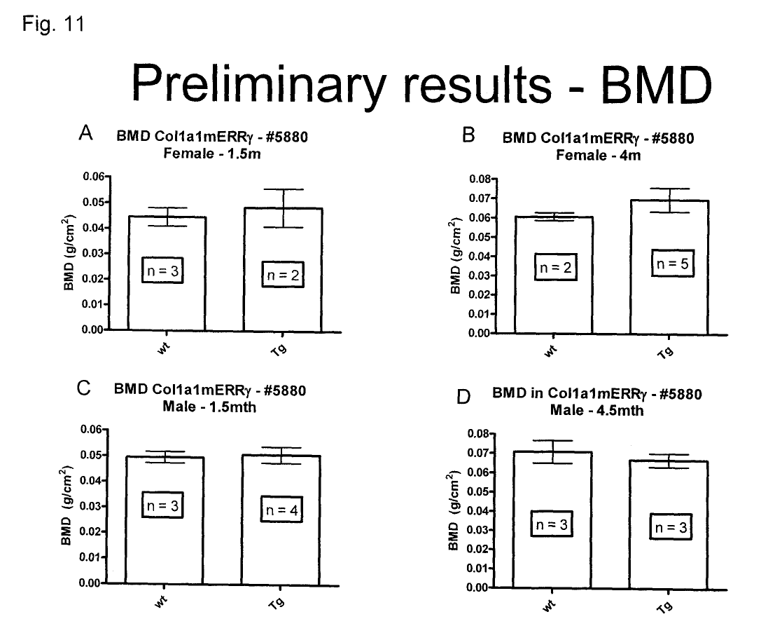

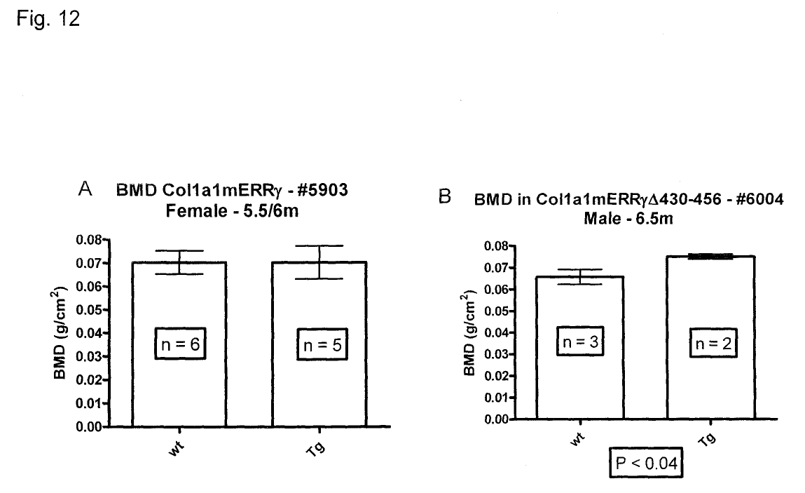

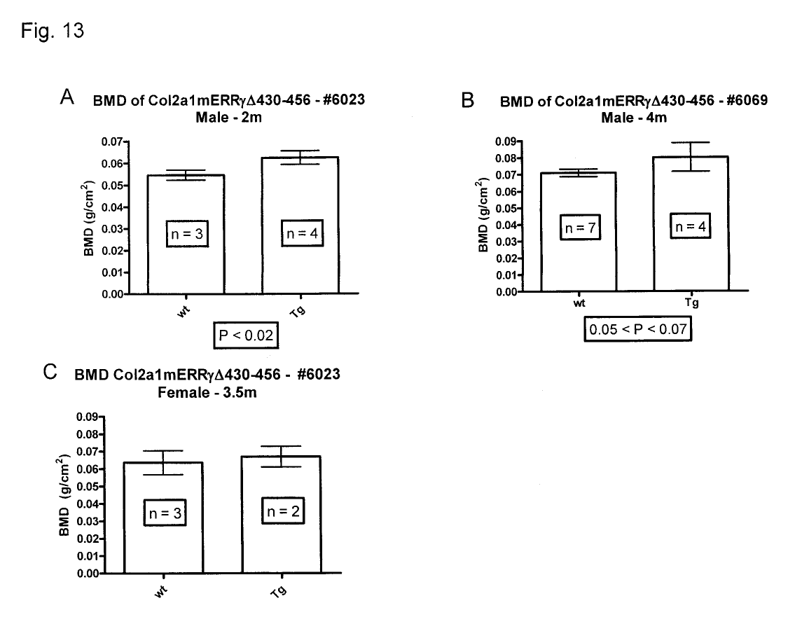

Osteoblast- or chondrocyte-specific ERRγ gain-of-function and loss-of-function transgenic mice (FIGS. 9 and 10), are viable and display no gross skeletal developmental or perinatal defects With reference to FIG. 9, ΔAF2 deletions of steroid receptors are usually considered loss-of-function mutations. On the osteopontin promoter, this is found to be true for ERRγ (i.e., ERRγ is an activator of the osteopontin promoter and ERRγΔAF2 (Δ430-456) fails to activate in osteoblastic cells). ERRγ knockout (Delatgen) is also being characterized. Measurements of bone mineral density were conducted on these postnatal transgenic mice (FIGS. 11, 12, and 13). Preliminary results show bone abnormalities when an ERRγ loss-of-function mutant is overexpressed in bone or cartilage. The preliminary data suggest that male and female mice are differentially sensitive to changes in ERRγ levels. ERRγ knockout mice are perinatal lethal, which may be due to cardiac defects, as previously observed by Alaynick et., 2007 in a different ERRγ knockout. There are no gross skeletal anomalies in the ERRγ knockout (data not shown).

From the above examples, at least the following conclusions may be drawn. In addition to ERRα (Bonnelye et al., 2001; Bonnelye et al., 2007), ERRγ is also expressed in osteoblasts and chondrocytes, and both of these receptors play functional roles in bone and cartilage formation. The functional activity of ERRγ in vitro appears opposite to the functional activity of ERRα. ERRγ appears to be a negative regulator of osteoblastogenesis and chondrogenesis. Preliminary analysis of ERRγ loss-of-function transgenic mice is consistent with this view. When ERRγ activity is reduced in either osteoblasts or chondrocytes, bone mineral density increases, but in a sex-dependent way; it appears that only the males are affected.

The present invention is not limited to the features of the embodiments described herein, but includes all variations and modifications within the scope of the description.

TABLE 1
Relative Expression of ERs and ERRs in Adult Tissues (ΔCT)

| | | | | | Mouse |
|---|---|---|---|---|---|
| | Rat brain | Rat kidney | Rat muscle | Rat femur* | cartilage** |
| ERRα | 3.38 ± 0.06 | 4.29 ± 0.07 | 3.69 ± 0.49 | 7.18 ± 0.08 | 8.16 ± 0.12 |
| ERRβ | 7.85 ± 0.12 | 6.80 ± 0.05 | 9.41 ± 0.40 | 16.44 ± 0.32 | 14.59 ± 0.34 |
| ERRγ | 5.10 ± 0.04 | 5.37 ± 0.2 | 5.28 ± 0.43 | 13.02 ± 0.18 | 13.17 ± 0.34 |
| ERα | 9.86 ± 0.24 | 5.20 ± 0.17 | 5.12 ± 0.52 | 11.31 ± 0.09 | 7.01 ± 0.13 |
| ERβ | 11.33 ± 0.16 | 18.30 ± 0.33 | 14.13 ± 0.54 | 20.92 ± 0.36 | 15.71 ± 0.21 |

*Femoral diaphyses, bone marrow removed.
**Epiphyses of distal femur:proximal tibiae, soft surrounding tissue gently dissected away.

Semi-quantitative real-time PCR was used to assess the expression level of ERRs and ERs in adult rat and mouse tissues. The results in Table 1 represent the ΔCt±SD of triplicate determinations from one biological sample set. ΔCt=Ctgene−CtL32; a higher number reflects lower expression.

TABLE 2
Relative Expression of ERs and ERRs in Established Cell Lines (ΔCT)

| | | | MC3T3-E1 | HeLa |
|---|---|---|---|---|
| | ROS17/2.8 | C5.18 | (subclone 26; | (human |
| | (Rat mature | (Rat | mouse | cervical |
| | osteoblastic | prechondrocyte | preosteoblastic | cancer |
| | cell line) | cell line) | cell line) | cell line) |
| ERRα | 7.80 ± 0.33 | 9.17 ± 0.11 | 8.79 ± 0.05 | 6.83 ± 0.06 |
| ERRβ | 15.28 ± 0.33 | 17.73 ± 0.61 | 15.40 ± 0.08 | 23.21 ± 0.53 |
| ERRγ | 17.48 ± 0.66 | 22.99 ± 0.21 | 19.36 ± 0.30 | 21.151 ± 0.34 |
| ERα | 14.28 ± 0.47 | 19.52 ± 0.27 | 11.32 ± 0.54 | 15.94 ± 0.30 |
| ERβ | 20.15 ± 0.47 | 23.67 ± 0.64 | 11.09 ± 0.13 | 18.46 ± 0.22 |

Semi-quantitative real-time PCR was used to assess the expression level of ERRs and ERs in rat, mouse and human cell lines. Results represent the ΔCt±SD of triplicate determinations from one biological sample set. ΔCt=Ctgene−CtL32; a higher number reflects lower expression.

### REFERENCES

Alaynick W A, Kondo R P, Xie W, He W, Dufour C R, Downes M, Jonker J W, Giles W, Naviaux R K, Giguere V, Evans R M. ERRgamma directs and maintains the transition to oxidative metabolism in the postnatal heart. Cell Metab. 2007 July; 6(1):13-24.
Aubin, J. E. and Triffitt, J. (2002). Mesenchymal stem cells and the osteoblast lineage. In Principles of Bone Biology, 2nd Edition, vol. 1 eds J. P. Bilezikian L. G. Raisz and G. A. Rodan), pp. 59-81. New York, N.Y.: Academic Press.
Blumberg, B. and Evans, R. M. (1998). Orphan nuclear receptors-new ligands and new possibilities. Genes Dev 12, 3149-3155.
Bonnelye, E. and Aubin, J. E. (2002). Differential expression of estrogen receptor-related receptor alpha and estrogen receptors alpha and beta in osteoblasts in vivo and in vitro. J Bone Miner Res 17, 1392-1400.
Bonnelye, E., Kung, V., Laplace, C., Galson, D. L. and Aubin, J. E. (2002). Estrogen receptor-related receptor alpha impinges on the estrogen axis in bone: potential function in osteoporosis. Endocrinology 143, 3658-3670.
Bonnelye, E., Merdad, L., Kung, V. and Aubin, J. E. (2001). The orphan nuclear estrogen receptor-related receptor (ERR) is expressed throughout osteoblast differentiation and regulates bone formation in vitro. J Cell Biol 153, 971-983.
Bonnelye, E., Zirngibl, R. A., Jurdic, P. and Aubin, J. E. (2007). The orphan nuclear estrogen receptor-related receptor-alpha regulates cartilage formation in vitro: implication of Sox9. Endocrinology. 148, 1195-1205.
Bridgewater, L. C., Lefebvre, V., and de Crombrugghe, B. (1998) Chodrocyte-specific enhancer elements in the Col1a2 gene resemble the Col2a1 tissue-specific enhancer. J Biol Chem 273(24), 14998-15006.
Busch, B. B., Stevens, W. C., Jr., Martin, R., Ordentlich, P., Zhou, S., Sapp, D. W., Horlick, R. A. and Mohan, R. (2004). Identification of a selective inverse agonist for the orphan nuclear receptor estrogen-related receptor alpha. J Med Chem 47, 5593-5596.
Chen, S., Zhou, D., Yang, C. and Sherman, M. (2001). Molecular basis for the constitutive activity of estrogen-related receptor alpha-1. J Biol. Chem. 276, 28465-28470.
Committee, T. N. R. N. (1999). The Nuclear Receptors Nomenclature Committee: A unified nomenclature system for the nuclear receptor superfamily. Cell 97, 161-163.
Conference, N.C.D. (1980). Steroid receptors in breast cancer: an NIH Consensus Development Conference, Bethesda, Md., Jun. 27-29, 1979. Cancer 46, 2759-2963.
Coward, P., Lee, D., Hull, M. V. and Lehmann, J. M. (2001). 4-hydrotamoxifen bind to and deactivates the estrogen-related receptor gamma. Proc Natl Acad Sci USA 98, 8880-8884.
Gaillard S, Grasfeder L L, Haeffele C L, Lobenhofer E K, Chu T M, Wolfinger R, Kazmin D, Koves T R, Muoio D M, Chang C Y, McDonnell DP. Receptor-selective coactivators as tools to define the biology of specific receptor-coactivator pairs. Mol Cell. 2006 Dec. 8; 24(5):797-803.
Giguere V. To ERR in the estrogen pathway. Trends Endocrinol Metab. 2002 July; 13(5):220-5.
Giguere, V., Yang, N., Segui, P. and Evans, R. M. (1988). Identification of a new class of steroid hormone receptors. Nature 331, 91-94.
Goater et al., (2000), J. Rheumatol., v. 27, pp. 983-989.
Greschik, H., Flaig, R., Renaud, J. P. and Moras, D. (2004). Structural basis for the deactivation of the estrogen-related receptor gamma by diethylstilbestrol or 4-hydroxytamoxifen and determinants of selectivity. J Biol Chem 279, 33639-33646.
Greschik, H., Wurtz, J. M., Sanglier, S., Bourguet, W., van Dorsselaer, A., Moras, D. and Renaud, J. P. (2002). Structural and functional evidence for ligand-independent transcriptional activation by the estrogen-related receptor 3. Mol Cell 9, 303-313.
Hodgson, S. F., Watts, N. B., Bilezikian, J. P., Clarke, B. L., Gray, T. K., Harris, D. W., Johnston, C. C., Jr., Kleerekoper, M., Lindsay, R., Luckey, M. M. et al. (2003). American Association of Clinical Endocrinologists medical guidelines for clinical practice for the prevention and treatment of postmenopausal osteoporosis: 2001 edition, with selected updates for 2003. Endocr Pract 9, 544-564.
Hong, H., Yang, L. and Stallcup, M. R. (1999). Hormone-independent transcriptional activation and coactivator binding by novel orphan nuclear receptor ERR3. J Biol Chem 274, 22618-22626.
Huppunen, J., Wohlfahrt, G. and Aarnisalo, P. (2004). Requirements for transcriptional regulation by the orphan nuclear receptor ERRgamma. Mol Cell Endocrinol 219, 151-160.
Johnston, S. D., Liu, X., Zuo, F., Eisenbraun, T. L., Wiley, S. R., Kraus, R. J. and Mertz, J. E. (1997). Estrogen-related receptor alpha 1 functionally binds as a monomer to extended half-site sequences including ones contained within estrogen-response elements. Mol Endocrinol 11, 342-352.
Kallen, J., Schlaeppi, J. M., Bitsch, F., Filipuzzi, I., Schilb, A., Riou, V., Graham, A., Strauss, A., Geiser, M. and Fournier, B. (2004). Evidence for ligand-independent transcriptional activation of the human estrogen-related receptor alpha (ERRalpha): crystal structure of ERRalpha ligand binding domain in complex with peroxisome proliferator-activated receptor coactivator-1alpha. J Biol Chem 279, 49330-49337.
Laflamme N, Giroux S, Loredo-Osti J C, Elfassihi L, Dodin S, Blanchet C, Morgan K, Giguere V, Rousseau F A frequent regulatory variant of the estrogen-related receptor alpha gene associated with BMD in French-Canadian premenopausal women. J Bone Miner Res. 2005 June; 20(6):938-44. Epub 2005 Feb 7.
Laganiere, J., Tremblay, G. B., Dufour, C. R., Giroux, S., Rousseau, F. and Giguere, V. (2004). A polymorphic autoregulatory hormone response element in the human estrogen-related receptor alpha (ERRalpha) promoter dictates peroxisome proliferator-activated receptor gamma coactivator-1alpha control of ERRalpha expression. J Biol Chem 279, 18504-18510.
Lefebvre, V., Zhou, G., Mukhopadhyay, K., Smith, C. N., Zhang, Z., Eberspaecher, H., Zhou, X., Sinha, S., Maity, S, N., and de Crombrugghe, B. (1996). An 18-base-pair sequence in the mouse proalpha1(II) collagen gene is sufficient for expression in cartilage and binds nuclear proteins that are selectively expressed in chondrocytes. Mol Cell Biol 16(8), 4512-4523.
Liu, D., Zhang, Z., Gladwell, W. and Teng, C. T. (2003). Estrogen stimulates estrogen-related receptor alpha gene expression through conserved hormone response elements. Endocrinology 144, 4894-4904.
Luo J, Sladek R, Carrier J, Bader J A, Richard D, Giguere V. Reduced fat mass in mice lacking orphan nuclear receptor estrogen-related receptor alpha. Mol Cell Biol. 2003 November; 23(22):7947-56.
Manolagas, S. C., Kousteni, S, and Jilka, R. L. (2002). Sex steroids and bone. Recent Prog Horm Res 57, 385-409.
Mootha, V. K., Handschin, C., Arlow, D., Xie, X., St Pierre, J., Sihag, S., Yang, W., Altshuler, D., Puigserver, P., Patterson, N. et al. (2004). Erralpha and Gabpa/b specify PGC-1alpha-dependent oxidative phosphorylation gene expression that is altered in diabetic muscle. Proc Natl Acad Sci USA 101, 6570-6575.
Nagy, A., Gertsenstein, M., Vintersten, K., Behringer, R. (Eds.) (2002) Manipulating the Mouse Embryo: A Laboratory Manual, Cold Spring Harbor Laboratory Press; 3 rd edition
Nam, K., Marshall, P., Wolf, R. M. and Cornell, W. (2003). Simulation of the different biological activities of diethylstilbestrol (DES) on estrogen receptor alpha and estrogen-related receptor gamma. Biopolymers 68, 130-138.
Panel, N.C.D. (2001). Osteoporosis Prevention, Diagnosis, and Therapy: NIH Consensus Development Panel on Osteoporosis Prevention, Diagnosis, and Therapy, Bethesda, Md., Mar. 27-29, 2000. JAMA 285, 785-795.
Richette, P., Corvol, M., Bardin, T., Luo, J., Sladek, R., Carrier, J., Bader, J. A., Richard, D. and Giguere, V. (2003). Estrogens, cartilage, and osteoarthritis. Joint Bone Spine 70, 257-262.
Riggs, B. L., Khosla, S, and Melton, L. J., 3rd. (2002). Sex steroids and the construction and conservation of the adult skeleton. Endocr Rev 23, 279-302.
Ritzen, E. M., Nilsson, 0., Grigelioniene, G., Holst, M., Savendahl, L. and Wroblewski, J. (2000). Estrogens and human growth. J Steroid Biochem Mol Biol 74, 383-386.
Robinson-Rechavi, M., Carpentier, A. S., Duffraisse, M. and Laudet, V. (2001). How many nuclear hormone receptors are there in the human genome? Trends Genet. 17, 554-556.
Rossert, J., Eberspaecher, H., and de Crombrugghe, B. (1995) Separate cis-acting DNA elements of the mouse pro-alpha 1(I) collagen promoter direct expression of reporter genes to different type I collagen-producing cells in transgenic mice J. Cell Biol. 129: 1421-1432.
Shi et al., (1997), Genomics, v. 44, pp. 52-60
Sims, N. A., Dupont, S., Krust, A., Clement-Lacroix, P., Minet, D., Resche-Rigon, M., Gaillard-Kelly, M. and Baron, R. (2002). Deletion of estrogen receptors reveals a regulatory role for estrogen receptors-beta in bone remodeling in females but not in males. Bone 30, 18-25.
Suetsugi, M., Su, L., Karlsberg, K., Yuan, Y. C. and Chen, S. (2003). Flavone and isoflavone phytoestrogens are agonists of estrogen-related receptors. Mol Cancer Res 1, 981-991.
Takashima-Sasaki K, Mori C, Komiyama M. Exposure of juvenile female mice to isoflavone causes lowered expression of estrogen-related receptor gamma gene in vagina. Reprod Toxicol. 2007 June; 23(4):507-512.
Tremblay G B, Bergeron D, Giguere V. 4-Hydroxytamoxifen is an isoform-specific inhibitor of orphan estrogen-receptor-related (ERR) nuclear receptors beta and gamma. Endocrinology. 2001 October; 142(10):4572-5. PMID: 11564725
Tremblay, G. B., Kunath, T., Bergeron, D., Lapointe, L., Champigny, C., Bader, J. A., Rossant, J. and Giguere, V. (2001). Diethylstilbestrol regulates trophoblast stem cell differentiation as a ligand of orphan nuclear receptor ERRbeta. Genes Dev 15, 833-838.
van der Eerden, B. C., Karperien, M. and Wit, J. M. (2003). Systemic and local regulation of the growth plate. Endocr Rev 24, 782-801.
van Lent et al., (2002), Osteoarthritis Cartilage, v. 10, pp. 234-243.
Vanacker, J. M., Pettersson, K., Gustafsson, J. A. and Laudet, V. (1999). Transcriptional targets shared by estrogen receptor-related receptors (ERRs) and estrogen receptor (ER) alpha, but not by ERbeta. Embo J 18, 4270-4279.
Willy, P. J., Murray, I. R., Qian, J., Busch, B. B., Stevens, W. C., Jr., Martin, R., Mohan, R., Zhou, S., Ordentlich, P., Wei, P. et al. (2004). Regulation of PPARgamma coactivator 1alpha (PGC-1alpha) signaling by an estrogen-related receptor alpha (ERRalpha) ligand. Proc Natl Acad Sci USA 101, 8912-8917.
Wluka, A. E., Cicuttini, F. M., Spector, T. D., Richmond, R. S., Carlson, C. S., Register, T. C., Shanker, G. and Loeser, R. F. (2000). Menopause, oestrogens and arthritis. Maturitas 35, 183-199.
Xie, W., Hong, H., Yang, N. N., Lin, R. J., Simon, C. M., Stallcup, M. R. and Evans, R. M. (1999). Constitutive activation of transcription and binding of coactivator by estrogen-related receptors 1 and 2. Mol Endocrinol 13, 2151-2162.
Yang, C. and Chen, S. (1999). Two organochlorine pesticides, toxaphene and chlordane, are antagonists for estrogen-related receptor alpha-1 orphan receptor. Cancer Res 59, 4519-4524.
Zhang, Z. and Teng, C. T. (2000). Estrogen receptor-related receptor alpha 1 interacts with coactivator and constitutively activates the estrogen response elements of the human lactoferrin gene. J Biol Chem 275, 20837-20846.
Zhang, Z. and Teng, C. T. (2007). Interplay between estrogen-related receptor alpha (ERRalpha) and gamma (ERRgamma) on the regulation of ERRalpha gene expression. Mol Cell Endocrinol. 264, 128-141.
Zhou, G., Garofalo, S., Mukhopadhyay, K., Lefebvre, V., Smith, C. N, Eberspaecher, H., and de Crombrugghe, B. (1995) A 182 by fragment of the mouse pro alpha 1(II) collagen gene is sufficient to direct chondrocyte expression in transgenic mice J Cell Sci 108: 3677-3684.
Zirngibl, R. A., Chan, J. M. S, and Aubin, J. E. (2006). The estrogen receptor related receptor a is a cell context dependent transcriptional activator of the mouse osteopontin promoter. J Bone Miner Res 21, S80.
Zirngibl, R. A., Chan, J. M. S, and Aubin, J. E. (2007). Estrogen receptor-related receptors regulate the osteopontin promoter in an isoform type and cell context dependant manner. IBMS abstract.
Zuercher W J, Gaillard S, Orband-Miller L A, Chao E Y, Shearer B G, Jones D G, Miller A B, Collins J L, McDonnell D P, Willson T M. Identification and structure-activity relationship of phenolic acyl hydrazones as selective agonists for the estrogen-related orphan nuclear receptors ERRbeta and ERRgamma. J Med. Chem. 2005 May 5; 48(9):3107-9.

1.-11. (canceled)

12. A method for promoting at least one of cartilage formation and bone formation in a tissue or cell in vitro comprising contacting the tissue or cell with an agent selected from the group consisting of:
(a) an ERRγ antagonist;
(b) a purified antibody which binds specifically to ERRγ protein;
(c) an antisense nucleotide sequence complementary to and capable of hybridizing to a nucleotide sequence encoding ERRγ protein; and
(d) an agent which reduces expression of the gene encoding ERRγ protein.

13. The method of claim 12 wherein the tissue is at least one of a cartilage biopsy and bone biopsy.

14. The method of claim 12 wherein the agent is an agent selected from the group consisting of diethylstilbestrol, 4-hydroxytamoxifen and 4-hydroxytoremifene.

15.-23. (canceled)

24. A method of promoting at least one of cartilage formation and bone formation in a mammal comprising administering to the mammal an effective amount of an agent selected from the group consisting of:
(a) an ERRγ antagonist;
(b) a purified antibody which binds specifically to ERRγ protein;
(c) an antisense nucleotide sequence complementary to and capable of hybridizing to a nucleotide sequence encoding ERRγ protein; and
(d) an agent which reduces expression of the gene encoding ERRγ protein.

25. The method of claim 24 wherein the agent increases proliferation of chondroprogenitor cells, osteoprogenitor cells, chondroblasts, and chondrocytes, and, osteoblasts, and osteocytes.

26. The method of claim 24 wherein the agent promotes differentiation of chondroprogenitor cells, osteoprogenitor cells, chondroblasts, and chondrocytes, and, osteoblasts, and osteocytes.

27. The method of claim 24 wherein the mammal suffers from a condition selected from the group consisting of cartilage loss, cartilage degeneration, cartilage injury, bone loss, bone degeneration and bone injury.

28. The method of claim 24 wherein the mammal suffers from arthritis.

29. The method of claim 24 wherein the mammal suffers from a disease selected from the group consisting of ankylosing spondylitis, childhood arthritis, chronic back injury, gout, infectious arthritis, osteoarthritis, osteoporosis, pagets's disease, polymyalgia rheumatica, pseudogout, psoriatic arthritis, reactive arthritis, reiter's syndrome, repetitive stress injury, and rheumatoid arthritis.

30. The method of claim 24 wherein the agent is administered systemically or orally.

31. The method of claim 24 wherein the agent is administered intra-articularly.

32. The method of claim 24 wherein the agent is an agent selected from the group consisting of diethylstilbestrol, 4-hydroxytamoxifen and 4-hydroxytoremifene.

33. A method of inhibiting at least one of cartilage formation and bone formation in a mammal comprising administering to the mammal an effective amount of an agent selected from the group consisting of:
(a) an ERRγ agonist;
(b) a substantially purified ERRγ protein;
(c) a nucleotide sequence encoding ERRγ protein or an effective portion thereof; and
(d) an agent which enhances expression of a gene encoding an ERRγ protein.

34. The method of claim 33 wherein the agent reduces proliferation of chondroprogenitor cells, osteoprogenitor cells, chondroblasts, and chondrocytes, and, osteoblasts, and osteocytes.

35. The method of claim 33 wherein the agent reduces differentiation of chondroprogenitor cells, osteoprogenitor cells, chondroblasts, and chondrocytes, and, osteoblasts, and osteocytes.

36. The method of claim 33 wherein the mammal suffers from chondrosarcoma, osteosarcoma, chondrodysplasia and osteodysplasia.

37. The method of claim 33 wherein the agent is administered systemically or orally.

38. The method of claim 33 wherein the agent is administered intra-articularly.

39. The method according to claim 33 wherein the agent is the phenolic acyl hydrazone GSK4716 or GSK9089.

40.-49. (canceled)

50. A pharmaceutical composition comprising a chondrogenesis inhibiting amount of an agent selected from the group consisting of:
(a) an ERRγ agonist;
(b) a substantially purified ERRγ protein;
(c) a nucleotide sequence encoding ERRγ protein or an effective portion thereof; and
(d) an agent which enhances expression of a gene encoding an ERRγ protein; and a pharmaceutically acceptable carrier.

51. A pharmaceutical composition comprising an osteogenesis inhibiting amount of an agent selected from the group consisting of:
(a) an ERRγ agonist;
(b) a substantially purified ERRγ protein;
(c) a nucleotide sequence encoding ERRγ protein or an effective portion thereof; and
(d) an agent which enhances expression of a gene encoding an ERRγ protein; and a pharmaceutically acceptable carrier.

52. A pharmaceutical composition comprising a cartilage formation promoting amount of an agent selected from the group consisting of:
(a) an ERRγ antagonist;
(b) a purified antibody which binds specifically to ERRγ protein;
(c) an antisense nucleotide sequence complementary to and capable of hybridizing to a nucleotide sequence encoding ERRγ protein; and (d) an agent which reduces expression of the gene encoding ERRγ protein and a pharmaceutically acceptable carrier.

53. A pharmaceutical composition comprising a bone formation promoting amount of an agent selected from the group consisting of:
(a) an ERRγ antagonist;
(b) a purified antibody which binds specifically to ERRγ protein;
(c) an antisense nucleotide sequence complementary to and capable of hybridizing to a nucleotide sequence encoding ERRγ protein; and
(d) an agent which reduces expression of the gene encoding ERRγ protein and a pharmaceutically acceptable carrier.

54. The pharmaceutical composition according to any one of claims 50 to 53, which is a solution, tablet, pill or suspension.


SEQUENCE LISTING (35 sequences)

SEQ ID NO: 1 - DNA, 23 bp, Artificial
  Chemically synthesized
cacaatgtcaaggagctggaagt

SEQ ID NO: 2 - DNA, 22 bp, Artificial
  Chemically synthesized
tctacgatggcttttcggttct

SEQ ID NO: 3 - DNA, 23 bp, Artificial
  Chemically synthesized
cacaatgtcaaggagctggaagt

SEQ ID NO: 4 - DNA, 22 bp, Artificial
  Chemically synthesized
tctacaatggcttttcggttct

SEQ ID NO: 5 - DNA, 20 bp, Artificial
  Chemically synthesized
caaggagctggaagtgctgc

SEQ ID NO: 6 - DNA, 20 bp, Artificial
  Chemically synthesized
cagctctttccacgatgcct

SEQ ID NO: 7 - DNA, 21 bp, Artificial
  Chemically synthesized
cctgcaaagccttcttcaaga

SEQ ID NO: 8 - DNA, 18 bp, Artificial
  Chemically synthesized
gcgtctccgcttggtgat

SEQ ID NO: 9 - DNA, 24 bp, Artificial
  Chemically synthesized
tcgagagatagtggtcaccatcag

SEQ ID NO: 10 - DNA, 22 bp, Artificial
  Chemically synthesized
cttccatccacacactctgcag

SEQ ID NO: 11 - DNA, 21 bp, Artificial
  Chemically synthesized
accgagagattgtggtcacca

SEQ ID NO: 12 - DNA, 22 bp, Artificial
  Chemically synthesized
catccacacgctctgcagtact

SEQ ID NO: 13 - DNA, 20 bp, Artificial
  Chemically synthesized
tgagatcaccaaacggaggc

SEQ ID NO: 14 - DNA, 18 bp, Artificial
  Chemically synthesized
gaactcggtcaaggcgca

SEQ ID NO: 15 - DNA, 23 bp, Artificial
  Chemically synthesized
gtttcctgaggtcaaggacttcc

SEQ ID NO: 16 - DNA, 19 bp, Artificial
  Chemically synthesized
agcctgtgtgacctgcagc

SEQ ID NO: 17 - DNA, 21 bp, Artificial
  Chemically synthesized
gacatcgcctctgggtatcac

SEQ ID NO: 18 - DNA, 20 bp, Artificial
  Chemically synthesized
gccgggcagctgtactctat

SEQ ID NO: 19 - DNA, 20 bp, Artificial
  Chemically synthesized
tgtgacttggctgaccgaga

SEQ ID NO: 20 - DNA, 21 bp, Artificial
  Chemically synthesized
tggaggaggctcatctggtct

SEQ ID NO: 21 - DNA, 21 bp, Artificial
  Chemically synthesized
acaaagcgcagacgtaaatcc

SEQ ID NO: 22 - DNA, 21 bp, Artificial
  Chemically synthesized
cgacctccacgtactctgtca

SEQ ID NO: 23 - DNA, 20 bp, Artificial
  Chemically synthesized
ggctgcgcaagtgttacgaa

SEQ ID NO: 24 - DNA, 21 bp, Artificial
  Chemically synthesized
catttcggccttccaagtcat

SEQ ID NO: 25 - DNA, 19 bp, Artificial
  Chemically synthesized
aagagggtgccaggctttg

SEQ ID NO: 26 - DNA, 17 bp, Artificial
  Chemically synthesized
tggagcgccagacgaga

SEQ ID NO: 27 - DNA, 25 bp, Artificial
  Chemically synthesized
ttggtgtgaagcaagatcactagag

SEQ ID NO: 28 - DNA, 23 bp, Artificial
  Chemically synthesized
gactagtaacagggctggcacaa

SEQ ID NO: 29 - DNA, 25 bp, Artificial
  Chemically synthesized
ttggtgtgaagcaagatcactagaa

SEQ ID NO: 30 - DNA, 23 bp, Artificial
  Chemically synthesized
gactagtaacagggctggcacaa

SEQ ID NO: 31 - DNA, 19 bp, Artificial
  Chemically synthesized
ccaacacctgggcaccttt

SEQ ID NO: 32 - DNA, 22 bp, Artificial
  Chemically synthesized
tctagcgatcttgcttcacacc

SEQ ID NO: 33 - DNA, 23 bp, Artificial
  Chemically synthesized
ggtggttgacgctgtccgtcagg

SEQ ID NO: 34 - DNA, 23 bp, Artificial
  Chemically synthesized
cctgacggacagcgtcaaccacc

SEQ ID NO: 35 - DNA, 23 bp, Artificial
  Chemically synthesized
caggtgctcggtacggtggctgt